(19) United States
(12) Patent Application Publication
(10) Pub. No.: US 2026/0306060 A1
(43) Pub. Date: Oct. 8, 2026

(54) SPINNING SINGLE ELEMENT ULTRASOUND TRANSDUCER AND FOCUSING TECHNOLOGY

(71) Applicant: Evident Vascular, Inc., San Jose, CA (US)

(72) Inventors: Rickard Clinton Loftman, Menlo Park, CA (US); Danielo B. Piazza, San Jose, CA (US); Patrick John Phillips, Los Altos, CA (US)

(21) Appl. No.: 19/474,878

(22) PCT Filed: Apr. 11, 2024
(86) PCT No.: PCT/US2024/024035

§ 371 Date: Oct. 13, 2025

Related U.S. Application Data
(60) Provisional application No. 63/497,962, filed on Apr. 24, 2023.

(51) Int. Cl. A61B 8/00; A61B 8/08; A61B 8/12; G16H 40/63

(52) U.S. Cl. CPC A61B 8/4461; A61B 8/0891; A61B 8/12; A61B 8/445; A61B 8/5207; G16H 40/63

(57) ABSTRACT

Systems and methods comprising a spinning, intravascular ultrasound transducer in a catheter are employed to optimize imaging via, for example, imaging frequency, acoustic aperture size, mechanical, physical, and/or electronic focusing. The effective focus for each individual scan line is modified, for example, by a phase sensitive combination of a plurality of neighboring scan line signals via properly adjusting amplitude, phase, and/or time axis adjustments.

### REFERENCE TO RELATED APPLICATIONS

This application claims the benefit of priority to U.S. Provisional Application No. U.S. App. 63/497,962 filed Apr. 24, 2023 titled “Spinning Single Element Ultrasound Transducer and Focusing Methods”, which is hereby incorporated by reference in its entirety.

### BACKGROUND

### Field

The present disclosure relates to the field of ultrasound, such as using ultrasound for diagnostic and imaging applications for medical purposes. Intravascular ultrasound (IVUS) imaging is provided in multiple embodiments that for example, includes a single transducer element that spins while imaging.

### Description of the Related Art

Ultrasound imaging transducers, including those used for intravascular imaging, emit and receive acoustic waves in order to produce an image based off reflections from objects near the ultrasound transducer, such as tissue.

### SUMMARY

Diagnosis and imaging of intravascular tissue and blood are critical in identifying irregularities, disease, and/or injury for medical treatment and can improve patient outcomes. Clarity of imaging from inside blood vessels such as is afforded by high resolution improves clinical utility, such as by the proper identification of lesions such as plaque (e.g., hard plaque, soft plaque, vulnerable plaque, calcified plaque, substantially non calcified plaque) or thrombus, facilitating relevant measurements and preparation for deploying stents, and is useful for planning of treatment by medical practitioners, e.g., physicians, surgeons, and/or medical technicians in various fields, such as vascular surgery and/or interventional cardiology throughout the body from the heart to the peripheral vasculature. In several embodiments, intravascular imaging-guided percutaneous coronary intervention for coronary artery lesions is achieved through the use of the IVUS imaging improvements described herein. In several embodiments, intravascular imaging-guided percutaneous intervention for peripheral vascular lesions is achieved through the use of the IVUS imaging improvements described herein. In several embodiments, intravascular imaging-guided percutaneous interventions in arteries and/or veins are achieved through the use of the IVUS imaging improvements described herein.

In several embodiments, intraluminal imaging is accomplished with the devices and methods described herein. Imaging and diagnosis of intraluminal (e.g., intravascular, cavity, digestive tract, esophagus, stomach, intestine, rectum, sinus, ureter, bladder, gynecological, etc.) tissue assists in identifying irregularities, disease, and/or injury for medical treatment to improve patient outcomes. Clarity of imaging is useful for proper identification of lesions, plaque (e.g., hard plaque, soft plaque, vulnerable plaque, calcified plaque, substantially non calcified plaque, fibro-fatty plaque), thrombus, calcium build up, dissections, and measurements of these abnormalities in several embodiments. IVUS imaging can be useful for planning treatment and guiding selection of therapies for interventional cardiology, vascular surgery, and interventional radiology throughout the body from the heart to the peripheral vasculature, such as for measurements of lesions and for guiding and assessing stent implantation. In several embodiments, the IVUS catheter is used in an artery, vein, blood vessel (e.g., carotid, subclavian, pulmonary, artic, renal, iliac, arteriovenous (AV) fistula, femoral, popliteal, tibial, etc.). In several embodiments, intraluminal imaging is accomplished with the devices and methods described herein. Imaging and diagnosis of intraluminal (e.g., intravascular, cavity, digestive tract, esophagus, stomach, intestine, rectum, pancreas, sinus, ureter, bladder, gynecological, etc.) tissue can assist in identifying irregularities, disease, and/or injury for medical treatment to improve patient outcomes. For example, endoscopes may be used with several features described herein. Transvaginal and other gynecological ultrasound devices may also include several features described herein. For embodiments in which EUS (endoscopic ultrasound) and other intraluminal imaging or imaging of other body cavities or organs is performed (and such imaging is not in a vessel), the features described herein for “IVUS” or “catheters” should be understood to apply to intraluminal (or other cavity/organ) catheters, probes, tubes, scopes and other such devices.

Imaging and diagnosis of intravascular or extravascular tissue is accomplished herein in several embodiments, including identification of irregularities. The term “irregularities” as used herein may be given its ordinary meaning and shall also include vascular or other malformations, constrictions, occlusions, dilations, disease, and injury. Irregularities may also include lesions, thrombus, aneurysm, dissection, plaque (e.g., hard plaque, soft plaque, vulnerable plaque, calcified plaque, substantially non-calcified plaque, fibro-fatty plaque), fistulas, tumors, neoplasia, gallstones, kidney stones, polyps, cysts, etc., and measuring morphology assessment wherein such irregularities are within vessels, other bodily lumens or other target sites. In several embodiments, irregularities may also include stent or balloon malapposition, dissection (e.g., post atherectomy, post balloon angioplasty, etc.), determining etiology of compression, under-expansion of a stent or other device, and issues with IVUS-guided sizing and grading of severity of an irregularity or under expansion of a stent, such as an iliac vein stent. Irregularities in the esophagus, stomach, and small intestine may be viewed using several embodiments described herein (e.g., through endoscopic ultrasound imaging).

In addition to the identification of irregularities, several embodiments herein are used to facilitate relevant measurements and preparation for deploying stents and/or balloons, and are useful for planning of treatment by medical practitioners.

After identification of irregularities, appropriate treatments may be used on the patient. For example, image-guided treatment of tumors, thrombus, plaque, etc. may be used in which imaging and therapeutic capabilities exist on a single device or multiple devices. Several embodiments include, for example, percutaneous coronary intervention for coronary artery lesions, intravascular imaging-guided percutaneous intervention for peripheral vascular lesions, and/or intravascular imaging-guided percutaneous interventions in arteries and/or veins. In several embodiments, the IVUS system is configured for optimized peripheral vascular procedures. In several embodiments, the IVUS system is configured for optimized peripheral vascular procedures and is not configured for coronary vascular procedures. In several embodiments, the IVUS system is configured for coronary procedures. In several embodiments, the IVUS system is configured for neurovascular procedures (including but not limited to cerebral vessels). In several embodiments, the IVUS system is configured for intravascular ultrasound-guided thrombectomy, including but not limited to mechanical thrombectomy. In some embodiments, the IVUS system is configured for ultrasound-guided pulmonary embolectomy. In several embodiments, simultaneous, real-time IVUS guidance is provided for procedures such as thrombectomy/embolectomy, stent placement, clot aspiration, other coronary or neurovascular procedures, etc.

Although several embodiments described herein described IVUS, the technology described herein are also used for intraluminal imaging (other than intravascular). For example, several embodiments are used for imaging, diagnosing, and/or providing an image-guided intervention in the digestive tract, esophagus, stomach, intestine, rectum, sinus, ureter, bladder, uterus, fallopian tubes, lungs, brain, etc. The systems and methods described herein may be used in conjunction with an endoscope rather than an IVUS catheter and support identification and diagnoses of gastrointestinal tumors, such as tumors in the intestines and/or biliary ducts. Similarly, the systems and methods described herein may be used to image a sinus cavity using IVUS.

In several embodiments, the technologies described herein, including the IVUS technologies for example, are used with other medical imaging systems (such as cardiac catheterization lab systems), to provide an integrated healthcare portfolio for cardiologists. An integrated or otherwise coordinated platform, in several embodiments, can improve workflow between various imaging systems, including for example, x-ray systems. In one embodiment, stent placement and other procedures are optimized using the IVUS technology described herein together with x-ray, external ultrasound and/or other non-IVUS technology.

In several embodiments, the IVUS technologies described herein are used with other catheter-based imaging procedures and/or non-catheter-based imaging procedures. Imaging procedures may include ultrasound, x-ray, computed tomography (CT), magnetic resonance imaging (MRI), positron emission tomography (PET), PET-CT, fluoroscopy, endoscopy, angiography, optical coherence tomography, intravital microscopy, 2D imaging, 3D imaging, etc. Several embodiments described herein utilize synchronized operation, imaging, and/or measurements from two, three or more imaging modalities (e.g., ultrasound, X-ray (including radiography, fluoroscopy, angiography, venography, etc.), magnetic resonance, PET scans, optical imaging (e.g., optical coherence tomography, light, laser imaging), etc.). Multi-modality synergies between IVUS and one or more additional imaging systems are achieved in several embodiments, including for example, enhanced visualization and image quality, decreased procedure time, increased precision in stent positioning and vessel measurements, improved workflow and reliability, and other benefits. Multi-modal systems including IVUS may be used, for example, to allow cardiologists to diagnose and/or treat vascular blockages and other defects that should, in turn, offer patients with improved cardiac outcomes, while reducing the overall cost burden to the healthcare system through efficient and effective integration with IVUS. A more robust image of vessel and organ structures (such as heart structure) can be obtained using various embodiments of the IVUS technologies described herein.

Standard use of acoustic imaging within blood vessels is often subject to certain limitations, including blurry images due to degraded effects from the catheter body itself due to standard design and limitations with fixed focusing. Standard single element technology can focus to a “natural focus” at the near-field/far-field transition, or to a single focus by adding a mechanical lens. Standard multi-element array implementations may experience similar blurry images due to the complexity and limitation of the electronics. Thus, several embodiments described herein are designed to improve (e.g., enhance) the quality of ultrasound images.

In several embodiments, optimization of ultrasound imaging from a spinning, rotating ultrasound transducer IVUS catheter is provided to produce improved near field focusing with a rotating single element ultrasound transducer to improve the lateral response of scan lines formed from individual passively focused pulse-echo events performed at various positions during rotation. In several embodiments, spinning (such as rotating) while imaging with ultrasound involves rotation or spinning about an axis (e.g., a longitudinal axis or length axis of a catheter, a lumen, a driveshaft, drive cable, drive coil, drivetrain, drive actuator). In several embodiments, ultrasound imaging takes place while rotating or spinning about a single axis in a single plane. In one embodiment, rotating does not solely comprise bending, such as bending about an axis, without also spinning or utilizing other rotational movements. In one embodiment, rotating does not comprise solely actuation, such as actuation that alters a linear axis. Imaging is improved by the phase sensitive sum of several neighboring scan lines that have each been judiciously adjusted in amplitude and/or phase, along with optional additional time axis shifting dependent on their depth and relative angular position, according to several embodiments. Methodologies for computing the scaling and delay and/or phase control per scan line vary due to specifics of the signal processing implementation. Weighting and time delay/phase adjustments will conform generally, in several embodiments, to linearizing the phase of the modified point spread function in azimuth. For example, the conjugate of phase seen in the multiplicity of signals being combined in this operation for each point in the image, for the case where these signals correspond to a point scatter at the current point of interest, may be determined and applied.

In the case of a narrowband or CW imaging system, according to several embodiments, the signal processing for transmit focus improvement reduces to convolution with a complex lateral filter, where the ideal phase profile of the filter is given by the complex conjugate of the imager point spread function at each given depth. This implementation is useful also in the case of a wideband imaging system according to several embodiments. Note that the control parameters of the focus improvement are implemented, pursuant to several embodiments herein, in a range dependent way so that the focus improvement may be maximized for all depths. The adjustments will vary, for example, as a function of depth. Improvements can be achieved in the case where catheter body material is affecting the passive natural focus adversely. For instance, if the catheter body has a detrimental effect on focusing in the far field, or if it has a defocusing effect for all depths, several embodiments herein may recover some performance, depending on the degree of original defocusing. In several embodiments, the opportunity for improvement is greater when the original focus is less ideal and there will be no (or minimal) advantage at a depth that is already perfectly focused. The adjustment of amplitude can be used to modulate the effect of the present method so that it can be disabled at a depth that is originally focused. Improvements can be achieved by several embodiments in the case where the directivity function is degraded by the continuous rotation of the device leading to misalignment of transmit and receive directivities.

Several embodiments are particularly advantageous because, unlike certain synthetic aperture systems with multiple element beamforming or with an array, the individual signals for some embodiments of a single rotating transducer described herein that are available for combination do not represent different individual sub-apertures.

According to various embodiments, the signals from the same single transducer do not have the same transmit and/or receive aperture specifications as with traditional transmit focus devices such as previous retrospective transmit focus devices. In several embodiments, the aperture implemented by a single rotating transducer (i) is not different between transmit and receive; (ii) produces only a line of round-trip focused data per pulse-echo event; and (iii) is rotating between each pulse echo event to affect the direction to which an ultrasound line corresponds. In several embodiments, both transmit and receive apertures are (i) complete and not subdivided or different with respect to the single signal produced by each pulse echo event and (ii) statically focused, unfocussed, or most likely imperfectly focused (meaning the energy contribution from each point along the aperture is not substantially equal phase). In several embodiments, the rotating transducer spins about an axis while imaging at rotations per second (rps) of 10-100 (e.g., 20-80, 30-50 rps).

According to various embodiments, a method for processing ultrasound data from a single rotating transducer situated within an elongated member comprises: (a) acquiring an original plurality of image lines wherein (i) each of the original plurality of image lines is acquired by a distinct pulse-echo emission and reception, and (ii) each of the original plurality of image lines corresponds to substantially one angular direction according to the physics of diffraction; (b) further processing the original plurality of lines to produce a modified plurality of lines with modified angular directivity characteristics by (i) a phase sensitive summation of several of the original plurality of image lines to produce each of a modified plurality of image lines, and (ii) a modification by phase rotation, time shifting, or both phase rotation and time shifting with or without additionally scaling amplitude prior to the phase sensitive summation, wherein the modification by phase rotation, time shifting, or both phase rotation and time shifting with or without additionally scaling amplitude is constructed so that the modified plurality of image lines will exhibit a more linear phase than the original plurality of lines about a direction corresponding to the location of a point target when imaged. In several embodiments, the rotating transducer spins about an axis while imaging at a rps of 10-100 (e.g., 20-80, 30-50 rps).

Additionally, in several embodiments, the modification prior to summation may be a function of imaging depth. The improvement of focusing may be achieved for point targets imaged at multiple depths in the image. The improvement of focusing may improve spatial resolution by narrowing the main peak of the response from the point target in the lateral dimension. In several embodiments, improvement of focusing may improve clutter rejection by lowering the response from the point target away from the main peak in the lateral dimension with a single rotating ultrasound transducer.

In other embodiments, also provided is use in case of interleaved line acquisition, for instance where near and far line segments are quickly acquired as an inner loop so the present operation is applied across like segments at different angles. For example in some embodiments, odd lines are combined to generate new odd lines and even lines are combined to generate new even lines. The odd and even lines may have some different designs or purpose such as interrogating different depth segments, or utilizing different frequencies, or providing for linear and non-linear response imaging signals.

The signal processing desired and/or required is feasible to produce high frame rate imaging within a computationally capable framework such as GPU accelerated software-based imaging pipeline according to one embodiment.

Among other benefits, spatial resolution improved by focusing is a fundamental aspect of image quality and is provided herein according to multiple embodiments. Although this benefit is particularly notable for the near field, near field is often clinically relevant as the transducer is often near or touching vessel layers of interest. In one embodiment, the improvements described herein may also facilitate adaptive Non Uniform Rotation Distortion (NURD) correction and other benefits for rotational devices.

In various embodiments, a method for processing ultrasound data for improved angular directivity and image clarity is provided that comprises image line modification via one or more of scaling of an amplitude, a phase rotation, and a time shift. In several embodiments, the image lines are acquired in a single plane. In several embodiments, modified image lines are within the single plane. For example, the modification of image line acquisition via one or more pulse-echo emissions and one or more pulse-echo receptions can have image lines that correspond to an angular directivity with one or more characteristics that are governed by diffraction. In various embodiments, modification involves scaling of amplitude, phase rotation, and/or time shift alone, or in combination. The method may be performed with an ultrasound transducer (e.g., single transducer, single element, rotating,). The ultrasound transducer may be connected to a driveshaft (e.g., drive cable, drive coil, drivetrain, drive actuator). In several embodiments, the ultrasound transducer images only while rotating or spinning about an axis (e.g., a longitudinal axis or length axis of a catheter, a lumen, a driveshaft, drive cable, drive coil, drivetrain, drive actuator). In several embodiments, ultrasound imaging takes place while rotating or spinning about a single axis in a single plane. In one embodiment, rotating does not comprise bending or actuation without spinning. For example, the ultrasound transducer and/or driveshaft may be located in a catheter, e.g., in a lumen or adjacent a wall, etc. The method can involve processing of the original plurality of image lines to produce a modified plurality of image lines with modified angular directivity characteristics. The method can involve a phase sensitive summation of at least two of the original plurality of image lines to produce each of the modified plurality of image lines with a more linear phase than the original plurality of image lines about a direction corresponding to a location of one or more isolated point targets when imaged.

In some embodiments, each of the original plurality of image lines corresponds to the angular directivity with a polar coordinate. The modification via phase rotation or the time shift can be a function of an imaging depth. Optional methods are provided achieving an improvement of image focusing for the one or more targets imaged at one or more depths in an image, or via improvement of image focusing that improves spatial resolution by narrowing a main peak of the pulse-echo reception from the one or more targets in a lateral dimension. In one embodiment, improvement of image focusing improves clutter rejection by lowering the response from the one or more targets away from a main peak in a lateral dimension.

In some embodiments, methods for improved focusing of an intravascular ultrasound catheter system include generating a plurality of ultrasonic signals, and receiving a plurality of backscatter ultrasonic signals that each correspond to one generated signal (e.g., one pulse-echo event). The backscatter ultrasonic signals can include one or more primary backscatter signals and one or more secondary backscatter signals. Imaging improvement may be accomplished by adjusting the one or more secondary backscatter signals based on the relationship to the one or more primary backscatter signals from which an isolated point target is imaged and then combining the primary backscatter signals and secondary backscatter signals to form a composite frame line and then generating an image based on composite frame lines formed according to an embodiment.

In various embodiments, an ultrasound device is provided that processes ultrasound data for improved angular directivity and image clarity. For example, an ultrasound catheter system configured for improved focus imaging can include a rotating ultrasound transducer that generates one or more ultrasonic signals with a processor that receives one or more backscatter ultrasonic signals (e.g., one or more primary backscatter signals, one or more secondary backscatter signals). In one embodiment, an ultrasound catheter system configured for improved focus imaging can include a rotating ultrasound transducer that generates one or more ultrasonic signals with a processor that receives one or more backscatter ultrasonic signals configured for adjusting the secondary backscatter signal(s) based on its relationship to the primary backscatter signal(s). In one embodiment, a system with a single imaging element processes only one ultrasound line at a time. Primary backscattered signals may be processed to generate an image. In another embodiment, each generated transmit signal produces one receive signal, and the one or more ultrasonic received signals may be considered peers, juxtaposed neighbors, until processed into an image The signal(s) may be processed individually, treating one signal as primary corresponding to a future line in an image and another juxtaposed signal or signals as secondary as the secondary signals are combined with the primary signal to improve imaging characteristics, such as sharpness and/or clarity. The device may optionally combine the primary backscatter signal(s) and secondary backscatter signal(s) to form a composite frame line in the direction of the primary image line and generate an improved clarity image based on the composite frame line for a plurality of primary lines.

The transducer can include a capture element. For example, a rotating single element ultrasound transducer can include a wideband capture element and/or a narrowband capture element. In some embodiments, a processor modifies the secondary backscatter signal by adjusting one or more of a time, a phase, or an amplitude based on a relationship to the primary backscatter signal. One or more primary and secondary backscatter signals can be summed with sensitivity to the phase via vector summation in a complex plane, such as in the case of baseband I and Q signal representation in one embodiment. In some embodiments, the phase sensitive summation could potentially occur at ultrasound frequency before any demodulation has occurred. Optionally, the processor is configured to adjust the secondary backscatter signal via convolution. In some embodiments, an ideal phase profile of the complex analytic filter is a complex conjugate of an imager point spread function at a particular depth. In various embodiments, the scan lines can include 2-400 scan lines (1, 5, 10, 16, 32, 45, 50, 64, 100, 128, 200, 256, 300, 400, and any values and ranges therein).

An ultrasound system comprising one ultrasound transducer element configured to rotate and generate a plurality of ultrasonic signals is provided. The transducer element is, in one embodiment, placed on or within a catheter. In several embodiments, a system comprises a processor configured to be placed in communication with the ultrasound transducer element, the processor configured to receive a plurality of backscatter ultrasonic signals. In one embodiment, processing of these signals includes phase sensitive combinations of a primary backscatter signal and one or more secondary backscatter signals, and adjusting the one or more secondary backscatter signals based on its relationship to the primary backscatter signal when an isolated point target is imaged. The primary backscatter signal and the one or more secondary backscatter signals may be combined to form a composite frame line. In one embodiment, this process is applied repeatedly to generate one or more images based on composite frame lines. The image may be displayed on a monitor, screen, head-mounted display, etc.

In general, several embodiments described herein provide an ultrasound transducer for positioning at an intravascular site with a catheter for acoustic imaging. Images and/or measurements of the vascular anatomy at the treatment site may be created by interrogating surrounding tissues with ultrasonic pulse echo events generated by the ultrasound transducer and creating cross-sectional, 360° images of the tissues based on a plurality of backscatter signals responsive to the ultrasonic pulses. In several embodiments, imaging is in one single plane. In one embodiment, a single spinning ultrasound element produces a single planar image. In one embodiment, imaging is multiplanar (e.g., multiple parallel planes) using a single element spinning transducer by moving the catheter with respect to tissue, such as during a pull back of the catheter to image along a length axis of a tissue. In one embodiment, imaging is not multiplanar.

The systems and methods described herein, according to several embodiments, are directed towards creating improved intravascular ultrasound imaging with a single, rotating transducer. More specifically, in several embodiments, the systems and methods described herein produce an intravascular ultrasound image with enhanced and improved focusing across a range of depths, or distances from the ultrasound transducer. In several embodiments, improved imaging is employed to optimize imaging. In one embodiment, a single ultrasound transducer (e.g., a single ultrasound transducer with only a single element, without multiple transducers, without multiple elements, without a plurality of transducers, without a plurality of elements, without an array of transducers and/or without an array of elements) is positioned at an intravascular site with a catheter for acoustic imaging. In one embodiment, an IVUS device creates 360-degree images with sequential frames with mechanical rotation of a single element transducer. In one embodiment, a 360-degree image is in one single plane. In one embodiment, a 360-degree image is not in multiple planes. Images and/or measurements of the vascular anatomy at the treatment site may be created by interrogating surrounding tissues from within the vasculature with ultrasonic pulse echo events directed along radial lines of changing angles by the rotation of the ultrasound transducer and creating an image of the tissues based on a plurality of backscatter signals responsive to the ultrasonic pulses. Vascular anatomy is imaged via interrogation from within the vasculature using acoustic, ultrasound pulse echo events direct to different angles from the one or more ultrasound transducers. In this way a series of images naturally suited to polar coordinates is produced. In one embodiment, IVUS images are formed from a plurality of radial ultrasound image lines aligned roughly to a 2D grid in a polar coordinate space orthogonal to the catheter length axis. In various embodiments, IVUS images are formed in a single plane orthogonal to the catheter length axis. In this way an annular image is presented to the user that represents a slice of anatomical information along the vasculature at the lengthwise position of the imaging acquisition unit.

In various embodiments, the angular or circumferential spatial resolution of IVUS imaging may be limited by the passive directivity function of the transducer given by the physics of diffraction. In one embodiment, this passive directivity involves no active means to control temporal delays or phases to actively focus or steer the ultrasound beam with a single transducer element, such as may be done with multi-element transducer arrays. The response of the imaging system to an isolated point target is called the point-spread function which can be collapsed to show only the lateral response when concerned specifically with that dimension.

In several embodiments, optimization of ultrasound imaging from a catheter is provided. In one embodiment, the lateral response characteristics of a passively focused device are controlled by modifying the imaging frequency, the acoustic aperture size (which is equal to the transducer element size for the case of a single element device), and/or altering the fixed focusing characteristics by means of a physical lens, such as moving the focal length from “infinity” (which can be produced by a flat transducer) to a shallower depth.

In one embodiment, improved imaging focusing is provided in the near field without a lens. In one embodiment, an IVUS device is provided with no true focusing lens, leading to what is referred to as “focus at infinity.” This infinite focus may be advantageous for imaging at deep distances, but this type of imaging may tend to have poor resolution in the near field in some embodiments. Changing the materials of the catheter body can somewhat improve the situation by approximating a shallower focal depth, but this can still be incomplete or suboptimal with a standard device construction. Advantageously, several embodiments described herein provide electronic systems and methods for improved imaging. The electronic features of said systems and methods may be used in addition to or instead of physical modifications (such as a mechanical lens to focus at a fixed depth). The electronic features can include, for example, using signal modifications to improve the effective focus for each individual scan line. This improved imaging can be accomplished, for example, by a phase sensitive combination of a plurality of neighboring scan line signals via properly adjusting amplitude, phase, and/or time axis adjustments. In one embodiment, the effective focus for each individual scan line is improved by a phase sensitive combination of a plurality of neighboring scan line signals via properly adjusting amplitude. In one embodiment, the effective focus for each individual scan line is improved by a phase sensitive combination of a plurality of neighboring scan line signals via properly adjusting phase. In one embodiment, the effective focus for each individual scan line is improved by a phase sensitive combination of a plurality of neighboring scan line signals via properly adjusting time axis adjustments.

In various embodiments, the intravascular ultrasound (IVUS) catheter images tissue. In various embodiments, the IVUS catheter is configured for improved imaging clarity of tissue and/or plaque (e.g., any one or more of hard plaque, soft plaque, vulnerable plaque, calcified plaque, substantially non calcified plaque). In various embodiments, the IVUS catheter is configured for improved imaging clarity of thrombus. In various embodiments, IVUS imaging includes improved focusing. In various embodiments, IVUS uses ultrasound for imaging only (without therapy). In various embodiments, IVUS uses ultrasound for therapy only (without imaging). In various embodiments, IVUS uses ultrasound for both imaging and therapy. According to several embodiments, one or more imaging technologies as described herein can be combined on the same catheter as one or more therapy elements, such as an integrated ultrasound imaging element located at or near the tip of (or otherwise along) a thrombectomy device. The thrombectomy device can also be a separate device that is delivered before, during or after the imaging device. Thrombectomy devices can be mechanical clot retrieval devices, clot aspiration devices, or a combination of clot retrieval and aspiration. Neurovascular, coronary and pulmonary clots are treated in several embodiments using the ultrasound imaging devices and methods disclosed herein together with (either integrated or separate) clot treatment devices. The clot treatment device can also include for example non-mechanical devices such as lytic or other drug delivery devices and energy delivery devices to disrupt/remove the clot or otherwise restore blood flow. Combinations of two, three or more therapies combined with the IVUS imaging technologies described herein are also provided (for example, ultrasonic or laser clot disruption with a lytic agent). The integrated IVUS and therapy catheter or probe can also be used, according to several embodiments, for restoring blood flow that is not caused by a clot.

In various embodiments, IVUS imaging occurs only while a single ultrasound transducer is rotating (e.g., spinning). In various embodiments, IVUS imaging occurs in a single plane while a single ultrasound transducer is rotating about a single axis. In one embodiment, imaging is not performed in multiple planes. In various embodiments, imaging is performed without multiple transducers, without multiple elements, without a plurality of transducers, without a plurality of elements, without an array of transducers and/or without an array of elements. In one embodiment, imaging is not performed while the ultrasound transducer is stationary.

Artificial intelligence and/or machine learning (AI/ML) are employed in some embodiments to enable and/or enhance image interpretation using the technologies described herein. In one embodiment, the system is matched for high definition (e.g., HD, UHD, HD+, etc.) image quality using acoustics and signal processing customized for peripheral vascular imaging with enhanced resolution and/or penetration. Several embodiments are configured for intravascular imaging with a platform that is optimized for peripheral and/or coronary vascular procedures that will enable improved image interpretation, intervention guidance, and enhance ease of use and improve overall usability to streamline intraprocedural and clinical workflow. In several embodiments, the system improves usability with a contemporary system featuring a simplified user-interface and enhanced total-system capabilities leveraging AI to streamline workflow and image interpretation. In several embodiments, the systems described herein, including for example the advanced intravascular ultrasound platform, leverage Artificial Intelligence (AI) to enable image interpretation, enhance total-system capabilities, and streamline workflows to maximize the clinical value. In some embodiments, advantageously, physicians will not need to integrate (e.g., cognitively integrate) imaging data spatially and temporally to fully interpret the clinical condition. Instead, systems according to several embodiments described herein can leverage the power of AI with generational advancements to go beyond single image interpretation. In several embodiments, the AI-powered engine, for example, may include a workstation that enhances image interpretation with a simplified workflow improving overall useability. Machine learning is used in several embodiments. In one embodiment, the AI-ready processing power is designed to support real time and on-demand image interpretation. The AI powered workstation can provide high end processing and an AI engine for advanced signal and image processing. In various embodiments, the native image data capture provides for superior image interpretation (e.g., border detection, identification and measurement of vessel size, vessel disease, dissection, plaque morphology, etc.). In several embodiments, the systems described herein provide simplified measurement via automated border detection (e.g., AI algorithms automatically identify borders of a lumen, vessel, tissue, lesion, plaque, etc.). In several embodiments, the system provides simplified measurement via semi-automated border detection (e.g., the user can manually adjust or modify automated AI algorithms that identify borders of a lumen, vessel, tissue, lesion, plaque, etc. with the border selection reconfigured based on user modifications). In one embodiment, AI plaque identification utilizes AI algorithms to automatically classify and identify types of plaque within the imaged area to provide user guidance on treatment options (e.g., using color coding, icons or text overlays can be used to indicate what type of condition, such as plaque, may be present for the selected image). In several embodiments, the data driven platform is designed to collect data, simplify image interpretation, with AI processing power to support real time and on-demand image interpretation and reduce user cognitive load to help (i) identify lumen size, (ii) visualize dissections, (iii) characterize disease morphology, (iv) locate and quantify stenosis, and/or (v) identify true lumen. In some embodiments, image interpretation is used to identify thrombus, thrombosis, clots, embolisms, plaque, calcium, tissue health, stent or balloon apposition, and/or stent or balloon “health” or condition. Image interpretation may involve imaging to evaluate quality and/or position of placement of an existing stent. Image interpretation can involve identifying position relative to lumen walls, determine level of and/or quality of tissue grown into and around the stent or balloon. In one embodiment, for example with a bioresorbable stent, image interpretation can involve (i) evaluating the amount of dissolving of the stent, (ii) determining if the dissolving of the stent is in accordance with expected decay patterns (e.g., determining whether the level of decay on one side of the stent similar to the other side of the stent, and if not, that may indicate a problem with stent placement, or if the stent is dissolving more rapidly than expected that could indicate the stent will not provide the tissue with the expected structural support). High-fidelity ultrasound data is used in one embodiment to drive improved image generation and image interpretation, with the option for leveraging artificial intelligence and/or machine learning. In various embodiments, catheters, devices, systems, and methods may be configured for use in performing edge-based machine learning computations associated with an image or image analysis using an artificial intelligence algorithm to identify a tissue border, plaque, calcium, thrombus, dissection, and/or stent apposition. In some embodiments, data, algorithms, AI and/or ML are used to obtain data from one or more sensors and provide feedback on operational aspects (such as imaging parameters) through a feedback loop (e.g., closed feedback loop/automated) or through user directed adjustments. In some embodiments, data, algorithms, AI and/or ML are used to obtain data from one or more images and provide feedback on operational aspects (such as imaging parameters and/or therapy) through a feedback loop (e.g., closed feedback loop/automated) or through user directed adjustments.

For example, image interpretation and measurement of luminal geometry (e.g., diameter, stenosis, peripheral interventions, coronary interventions, atherectomy, lithotripsy, intravascular lithotripsy (IVL), balloon placement, stent placement, venous procedures, below the knee (BTK) procedures, AV Fistula, and other procedures are performed with embodiments described herein. Procedures can be performed by interventional cardiologists, radiologists, and/or vascular surgeons in hospitals, physician offices, Office Based Labs (OBL), and/or Ambulatory Surgery Centers (ASC). In several embodiments, systems described herein can be used efficiently in the hospital and OBL/ASC.

In some embodiments, advantageously, physicians will not need to integrate (e.g., cognitively integrate) imaging data spatially and temporally to fully interpret the clinical condition. Instead, systems according to several embodiments described herein can leverage the power of AI with generational advancements to go beyond single image interpretation. The AI-powered engine, for example, enhances image interpretation with a simplified workflow improving overall useability. Machine learning is used in several embodiments. In one embodiment, the AI-ready processing power is designed to support real time and on-demand image interpretation. The AI powered workstation can provide high end processing and an AI engine for advanced signal and image processing. In various embodiments, the native image data capture provides for superior image interpretation (e.g., border detection, identification and measurement of vessel size, vessel disease, dissection, plaque morphology, etc.). In several embodiments, the systems described herein provide simplified measurement via automated border detection (e.g., AI algorithms automatically identify borders of a lumen, vessel, tissue, lesion, plaque, etc.). In several embodiments, the system provides simplified measurement via semi-automated border detection (e.g., the user can manually adjust or modify automated AI algorithms that identify borders of a lumen, vessel, tissue, lesion, plaque, etc. with the border selection reconfigured based on user modifications). In one embodiment, AI plaque identification utilizes AI algorithms to automatically classify and identify types of plaque within the imaged area to provide user guidance on treatment options (e.g., using color coding, icons or text overlays can be used to indicate what type of condition, such as plaque, may be present for the selected image). In several embodiments, the data driven platform is designed to collect data, simplify image interpretation, with AI processing power to support real time and on-demand image interpretation and reduce user cognitive load to help (i) identify lumen size, (ii) visualize dissections, (iii) characterize disease morphology, (iv) locate and quantify stenosis, and/or (v) identify true lumen. In some embodiments, image interpretation is used to identify thrombus, plaque, calcium, tissue health, stent or balloon apposition, and/or stent or balloon “health” or condition. Image interpretation may involve imaging to evaluate quality and/or position of placement of existing stent. Image interpretation can involve identifying position relative to lumen walls, determine level of and/or quality of tissue grown into and around the stent or balloon. In one embodiment, for example with a bioresorbable stent, image interpretation can involve (i) evaluating the amount of dissolving of the stent, (ii) determining if the dissolving of the stent is in accordance with expected decay patterns (e.g., determining whether the level of decay on one side of the stent similar to the other side of the stent, and if not, that may indicate a problem with stent placement, or if the stent is dissolving more rapidly than expected that could indicate the stent will not provide the tissue with the expected structural support).

In several embodiments, the technologies described herein, including the IVUS technologies for example, are used with other medical imaging systems (such as cardiac catheterization lab systems), to provide an integrated healthcare portfolio for cardiologists. An integrated or otherwise coordinated platform, in several embodiments, can improve workflow between various imaging systems, including for example, x-ray systems. In one embodiment, stent placement and other procedures are optimized using the IVUS technology described herein together with x-ray, external ultrasound and/or other non-IVUS technology.

In several embodiments, the IVUS technologies described herein are used with other catheter-based imaging procedures and/or non-catheter-based imaging procedures. Imaging procedures may include ultrasound, x-ray, computed tomography (CT), magnetic resonance imaging (MRI), positron emission tomography (PET), PET-CT, fluoroscopy, endoscopy, angiography, optical coherence tomography, intravital microscopy, 2D imaging, 3D imaging, etc. Several embodiments described herein utilize synchronized operation, imaging, and/or measurements from two, three or more imaging modalities (e.g., ultrasound, X-ray (including radiography, fluoroscopy, angiography, venography, etc.), magnetic resonance, PET scans, optical imaging (e.g., optical coherence tomography, light, laser imaging), etc.). Multi-modality synergies between IVUS and one or more additional imaging systems are achieved in several embodiments, including for example, enhanced visualization and image quality, decreased procedure time, increased precision in stent positioning and vessel measurements, improved workflow and reliability, and other benefits. Multi-modal systems including IVUS may be used, for example, to allow cardiologists to diagnose and/or treat vascular blockages and other defects that should, in turn, offer patients with improved cardiac outcomes, while reducing the overall cost burden to the healthcare system through efficient and effective integration with IVUS. A more robust image of vessel and organ structures (such as heart structure) can be obtained using various embodiments of the IVUS technologies described herein.

Co-Registration of Images: In several embodiments, an IVUS system provides co-registration data to provide a 1:1 co-location identification enabling therapeutic precision. Advantageously, several embodiments described herein can work collaboratively with, or independently from, co-registration with another imaging modality such as fluoroscopy, in which a software algorithm tracks radio-opaque (RO) catheter marker(s) or a RO transducer throughout a continuous fluoroscopy recording. Co-registration with angiography may be used, for example, to determine 3D shape of vessel, lumen and lesion, including lesion length, efficient stent selection, efficient location for stent landing zone, in an attempt to shorten procedure time, decrease contrast use, and make practitioners more comfortable with IVUS. Described herein, are several embodiments that accomplish one or more of these benefits with, or without, co-registration.

Plug and Play Catheter: According to several embodiments, catheters are optimized for vascular imaging and configured for superior pushability, tracking, and crossing for arterial and venous vasculature. In several embodiments, catheters have superior pushability to avoid kinking with sufficient column strength to advance the catheter through tortuous bends and occlusions in vasculature without buckling, over bending, or collapsing anywhere along the catheter (e.g., ability to cross an occlusion or constriction). In several embodiments, catheters have superior tracking for the ability of the catheter to follow a guidewire through tortuous bends in vasculature, having sufficient flexibility and strength to move along and advance along a guidewire to target locations within the vasculature. In several embodiments, catheters have superior crossing capabilities to cross occlusions, restrictions and constrictions within the vasculature, such as at sites with tissue blockage (e.g., stenoses, etc.) and/or implant blockages (such as stents, balloons, etc.). The systems described herein, such as an IVUS catheter, can include a rotational design that is plug and play and for example, can allow the catheter to be taken out of the sterile package and prepared for use without the need to flush the device. In one embodiment, the catheter has a single rotational ultrasound element. In several embodiments, the catheter includes an encapsulated coupling medium (e.g., coupling medium, medium, liquid, fluid, gel, etc.) that supports the spinning imaging core inside the catheter jackets. In one embodiment, the IVUS catheter is a plug and play catheter with a rotational IVUS design peripheral disposable imaging catheter with a full length of 280 cm with a working length of 150 cm compatible with a 0.014″ guidewire and a 5F sheath, allowing the IVUS catheter proximal connector to attach to the CIM outside of the sterile field. In one embodiment, the IVUS catheter is a plug and play catheter is a peripheral disposable imaging catheter with a full length of 250 cm with a working length of 110 cm compatible with a 0.035″ guidewire and an 8F sheath, allowing the IVUS catheter proximal connector to attach to the CIM outside of the sterile field. Connecting outside of the sterile field optionally avoids having to drape a cabled motor unit inside of the sterile field. In several embodiments, the high definition (e.g., HD, ultra high definition (UHD), UHD, HD+, etc.) imaging uses acoustic pulse echoes with a matched excitation frequency spectrum for optimal penetration, ultra high resolution, and high definition image quality. In several embodiments, the system is optimized for peripheral vascular imaging, coronary imaging, or both. The imaging core spins inside the polymer jacket using an inner drive shaft connected through a proximal hub/connector in one embodiment. The hub connector is optionally attached to the CIM after removal from its sterile catheter package. The transducer located at the distal tip of the imaging core can rotate at between 1500-4000 rpm and receives echoes for processing into a circular image on the tablet display. The length of the catheter can be 8 to 10 feet long. In various embodiments the catheter can be taken out of the sterile package and prepared for use without the need to flush the device. The lengths can allow the catheter proximal connector to connect to the CIM outside of the sterile field. Connecting outside of the sterile field avoids having to drape a cabled motor unit inside of the sterile field in one embodiment. The catheter can include non-volatile memory that includes unique catheter identification, usage data, and calibration for optimal imaging performance. Calibration may include data related to measurements of electrical impedance versus frequency, acoustic sensitivity versus frequency, and/or beam profile data that is specific to the individual device according to some embodiments.

Catheter Interface Module (CIM): A CIM is a hardware interface between the system cable from a workstation and disposable catheters according to several embodiments. In several embodiments, the CIM provides the system (e.g., IVUS system) with a rotational drive and ultrasound signal processing functions. Custom electronics may control the motor that rotates the imaging core inside the catheter. The electronics may also transmit and receive ultrasound signals between the spinning transducer within the distal end of the catheter tip and the custom printed circuit board inside the workstation. In various embodiments, the CIM provides an interface to read and write non-volatile memory in, for example, the catheter. Memory can be used to calibrate an ultrasound transducer for each unique catheter. In one embodiment, catheter memory may be used to select the appropriate system configuration to achieve the best possible imaging performance. The CIM may transmit ultrasound signals, sensor data, and/or catheter information from a catheter to a workstation and/or at least one user interface device (e.g., tablet, computer, etc.), which may store the transmitted information in a non-volatile memory in one or more locations. In one embodiment, the CIM is mounted on a bed rail outside a sterile field. In several embodiments, the CIM is embedded or integrated into imaging control equipment, a workstation, a housing, a table, a bed, a pedestal, a platform, and/or a cart. In several embodiments, the CIM is located outside a sterile field. One or more ports to allow for seamless connection between IVUS systems and other imaging modalities are provided in several embodiments.

As used in the summary above, as well the description below, where a device or a method “comprises” or “includes” (the two being interchangeable) certain features or steps, such device or method may also “consist essentially of” some of those features or steps if identified as such (i.e. recites “consists essentially of” in the claims).

The technology described herein is used in several embodiments with flush-less, encapsulated seal IVUS catheters to reduce air bubbles and eliminate need to flush acoustic coupling fluid including the technology described in U.S. Ser. No. 63/459,312 entitled Systems and Methods for Flush-Less Intravascular Ultrasound Catheter and U.S. Ser. No. 63/546,091 entitled Systems and Methods for Flush-Less Intravascular Ultrasound Catheter (and the PCT application claiming priority thereto and filed April 11, 2024); manually assisted pullback for spatial alignment measurements, voice control, position sensors (e.g., encoder) including the technology described in U.S. Ser. No. 63/531,266 entitled Systems and Methods of Manually Assisted Pullback for Spatial Alignment Lengthwise Measurements in Intravascular Ultrasound Imaging; and ultrasound imaging systems and components, voice control, and artificial intelligence algorithms including the technology described in U.S. Ser. No. 63/546,058 entitled Systems and Methods for Intravascular Ultrasound, all herein incorporated by reference in their entirety into the disclosure.

### BRIEF DESCRIPTION OF THE DRAWINGS

The following drawings are for illustrative purposes only and show non-limiting embodiments. Features from different figures may be combined in several embodiments.

FIG. 1 is an example block diagram of a rotational intravascular ultrasound system according to one embodiment;

FIG. 2 illustrates an example process for generating a processed intravascular ultrasound image according to one embodiment;

FIG. 3 illustrates an example process for generating a processed intravascular ultrasound image according to one embodiment;

FIG. 4A depicts a contour plot illustrating the angular sensitivity of a continuous wave imaging system according to one embodiment;

FIG. 4B depicts a contour plot illustrating the angular sensitivity with improvement of a continuous wave imaging system according to one embodiment;

FIG. 4C depicts a contour plot illustrating the angular sensitivity of a continuous wave imaging system with an unfavorable choice of speed of sound in the catheter body according to one embodiment;

FIG. 4D depicts a contour plot illustrating the correction of a continuous wave imaging system with poor angular resolution according to one embodiment.

FIG. 4E depicts a contour plot illustrating a computed phase adjustment as a function position relative to a central line according to one embodiment.

### DETAILED DESCRIPTION

According to several embodiments, the systems and methods described herein are directed towards an improved intravascular ultrasound imaging with a rotating transducer. In several embodiments, the ultrasound imaging is performed only while rotating or spinning about an axis (e.g., a longitudinal axis of a catheter, a lumen, a driveshaft, drive cable, drive coil, drivetrain, drive actuator). In several embodiments, ultrasound imaging takes place while rotating or spinning about a single axis in a single plane. In one embodiment, rotating does not comprise bending, such as bending about an axis, without also spinning. In one embodiment, rotating does not comprise actuation, such as actuation orthogonal to an axis, such as a linear axis, a catheter body, without also spinning. For example, the systems and methods described herein produce an intravascular ultrasound image that with enhanced and improved focusing across a range of depths, or distances from the ultrasound transducer. Typically, rotational intravascular ultrasound acoustic apertures can be unfocused, or slightly focused/de-focused by refraction through the catheter body. In various embodiments, improved imaging focusing is achieved when spinning in 360-degree rotations within the catheter for sequential imaging frames via mechanical rotation. NURD may be a risk to practical application for rotational devices if present.

In various embodiments, the systems and methods described herein produce an intravascular ultrasound image with enhanced and improved focusing across a range of depths, or distances from the ultrasound transducer. In several embodiments, improved imaging is employed to optimize imaging. In one embodiment, a single ultrasound transducer (e.g., with only a single element, without multiple elements, without a plurality of elements, and/or without an array of elements) is positioned at an intravascular site with a catheter for acoustic imaging. In various embodiments, IVUS imaging occurs only while a single ultrasound transducer is rotating. In various embodiments, IVUS imaging occurs in a single plane while a single ultrasound transducer is rotating about a single axis (e.g., a length axis of a catheter, lumen, or drive shaft). In one embodiment, imaging is not performed in multiple planes. In various embodiments, imaging is performed without multiple transducers, without multiple elements, without a plurality of transducers, without a plurality of elements, without an array of transducers and/or without an array of elements. In one embodiment, imaging is not performed while the ultrasound transducer is stationary. In one embodiment, an IVUS device creates 360-degree images with sequential frames with mechanical rotation of a single element transducer. Images and/or measurements of the vascular anatomy at the treatment site may be created by interrogating surrounding tissues from within the vasculature with ultrasonic pulse echo events directed along radial lines of changing angles by the rotation of the ultrasound transducer and creating an image of the tissues based on a plurality of backscatter signals responsive to the ultrasonic pulses. In one embodiment, a 360-degree image is in one single plane. In one embodiment, a 360-degree image is not in multiple planes. In one embodiment, a polar coordinate system is employed. Vascular anatomy is imaged via interrogation from within the vasculature using acoustic, ultrasound pulse echo events direct to different angles from the ultrasound transducer (such as a single, spinning transducer). In this way a series of images naturally suited to polar coordinates is produced. In various embodiments, the IVUS imaging occurs in a single plane while a single ultrasound transducer is rotating about a single axis (e.g., a length axis of a catheter, lumen, or drive shaft) for improved imaging clarity and/or sharpness of tissue and/or plaque (e.g., any one or more of hard plaque, soft plaque, vulnerable plaque, calcified plaque, substantially non calcified plaque). In various embodiments, the IVUS imaging occurs in a single plane while a single ultrasound transducer is rotating about a single axis for improved imaging clarity of thrombus.

In various embodiments, the angular or circumferential spatial resolution of IVUS imaging may be limited by the passive directivity function of the transducer given by the physics of diffraction. In various embodiments, the IVUS system has an imaging axial resolution of 10-500 μm (e.g., 10, 20, 30, 40, 50, 60, 70, 80, 90, 100, 125, 150, 175, 200, 225, 250, 275, 300, 325, 350, 375, 400, 425, 450, 475, 500 μm and any values and ranges therein). In various embodiments, the IVUS system has an imaging lateral resolution of 10-500 μm (e.g., 10, 20, 30, 40, 50, 60, 70, 80, 90, 100, 110, 125, 145, 150, 175, 185, 200, 215, 225, 250, 275, 290, 300, 325, 350, 375, 400, 425, 450, 475, 500 um and any values and ranges therein). In various embodiments, the IVUS system has an imaging lateral resolution of 2-20 degrees (e.g., 2, 3, 4, 5, 6, 7, 8, 9, 10, 11, 12, 13, 14, 15, 16, 17, 18, 19, and 20 degrees and any values and ranges therein). In various embodiments, rotating or spinning a single ultrasound transducer about a single axis while imaging improves the clarity and sharpness of the imaging by 5-200% or more. In various embodiments, IVUS imaging resolution is improved 5-200% (e.g., 5, 10, 15, 20, 25, 30, 35, 40, 45, 50, 55, 60, 65, 70, 75, 80, 85, 90, 95, 100, 110, 120, 130, 140, 150, 160, 170, 180, 190, 200% and any values and ranges therein). In one embodiment, passive directivity involves no active means to control temporal delays or phases to actively focus or steer the ultrasound beam with a single transducer element, such as may be done with multi-element transducer arrays. The response of the imaging system to an isolated point target is called the point-spread function which can be collapsed to show only the lateral response when concerned specifically with that dimension.

Certain factors that are under control of a device designer to optimize the lateral response characteristics of such a passively focused device include the imaging frequency (such as, for example, in the range of 1-90 MHz (e.g., 1-10, 10-15, 10-20, 10-25, 10-30, 10-40, 15-30, 15-35, 15-40, 15-50, 15-60, 15-70, 20-30, 30-40, 40-50, 20-25, 20-30, 20-40, 20-45, 20-50, 25-35, 25-40, 25-45, 25-50, 30-45, 30-50, 40-45, 45-50, 50-60, 50-70, 50-80, 55-75, 55-65, 60-70, 60-90, 70-80, 70-90 MHz and any values and ranges therein) In one embodiment, the device has a frequency of 60 MHz. In several embodiments, lateral response characteristics of such a passively focused device are optimized, such as the acoustic aperture size (which is equal to the transducer element size for the case of a single element device), and potentially altering the fixed focusing characteristics by means of a physical lens, such as moving the focal length from “infinity” (which can be produced by a flat transducer) to a shallower depth. In various embodiments, fixed focus depths are in a range of 1-10 mm (e.g., 1.0, 1.1, 1.2, 1.3, 1.4, 1.5, 1.7, 1.8, 2.0, 2.4, 2.5, 2.7, 2.8, 2.9, 3.0, 3.1, 3.2, 3.3, 3.4, 3.5, 3.7, 4.0, 4.5, 5.0, 5.5, 6.0, 6.5, 7.0, 7.5, 8.0, 8.5, 9.0, 9.5, and 10.0 mm and any values or ranges therein. In one embodiment, the fixed focus depth is 2.5 mm-3.5 mm (e.g., 2.5, 2.6, 2.7, 2.8, 2.9, 3.0, 3.1, 3.2, 3.3, 3.4, 3.5 mm and any values or ranges therein).

With standard single element rotating IVUS devices, focusing of the aperture can often be sub-optimal, especially in the near field. Standard devices typically have no true focusing lens, leading to what is referred to as “focus at infinity.” This infinite focus may be too deep or distant for the targeted clinical imaging requirements which results in poor spatial resolution in the near field. In some embodiments, favorable material choices of the catheter body can improve the situation by approximating a shallower focal depth, but this can still be incomplete or suboptimal with standard device construction. In one embodiment, because any physical mitigation is passive, it cannot actively adjust focus as a function of imaging depth. Standard intravascular ultrasound systems can produce sub-optimal blurry imaging, generally relying on a natural focus at the near-field/far-field transition. The angular or circumferential spatial resolution of intravascular ultrasound imaging can also be characterized by the imaging system's lateral sensitivity function (e.g., via the imager's point-spread function). Certain embodiments rely on solutions such as mechanical lenses which enable an unfocused single-element transducer intravascular ultrasound system to focus on a single depth. With standard intravascular ultrasound systems, it is often not possible to focus individual lines at more than one depth with a single element, and the near field especially suffers.

In some embodiments, an intravascular ultrasound (IVUS) system includes a catheter body, an ultrasound imaging transducer (e.g., with a single transduction element), a rotational driveshaft, hardware, one or more connectors, and software run via one or more processors. The one or more processors may be configured to receive a plurality of backscatter ultrasonic signals which may include a primary backscatter signal and one or more secondary backscatter signals. The one or more processors may also be configured to adjust the one or more secondary backscatter signals based on its relationship to the primary backscatter signal for the case of a point target at the point of interest; combine the primary backscatter signal and the one or more secondary backscatter signals to form a composite frame line; and generate an image based on the composite frame line.

The systems and methods described herein are directed towards creating improved intravascular ultrasound imaging. More specifically, the systems and methods described herein produce an intravascular ultrasound image that with enhanced and improved focusing across a range of depths, or distances from a rotating ultrasound transducer. In several embodiments, improved imaging is employed to optimize imaging.

In various embodiments, ultrasonic pulse-echo images are formed from a plurality of pulse-echo events. A pulse echo event includes a transmission phase where a typically short pulse of acoustic energy is emitted followed by a reception phase where energy returning from echoes from the interrogated medium are sensed and processed. The recorded time of flight of a typically short high-bandwidth pulse provides the ability to discriminate the depth from which an echo originates. In one embodiment, this corresponds to the radial dimension of polar coordinate image data formation. The ability to discern the direction from which echo originates is governed by acoustic diffraction. This corresponds to the azimuthal dimension of polar coordinate data image formation and also the elevation or out of plane dimension. In one embodiment, the diffraction pattern is controlled principally by frequency content and aperture. The aperture is the surface used to create and sense the acoustic energy, such as that defined by the face of a single transducer element. In various embodiments, a lens on the aperture controls the shape of wavefronts by differentially delaying signals associated with different parts of the aperture which affects the diffraction pattern by steering and focusing. In the case of a passively focused aperture, the lens is physical or mechanical such as a material of distinct speed of sound and varying thickness.

In one embodiment, focus control of single element IVUS devices is constrained without a lens. An aperture with no lens has effectively an ideal focus performance at the depth of the near-field to far-field transition (called the “natural focus”) and beyond. In one embodiment, a single element IVUS relies on this natural focusing achieved with no lens. However, different materials are typically in the acoustic path, such as a polymer jacket which can act as a lens whether desired or not. Focus performance may be improved or degraded by the choice of geometry and material specifications of the device. In one embodiment, a high performance mechanical lens may be used, but is typically cost prohibitive. Standard rotational IVUS acoustic apertures are unfocussed, in the sense that there is no specific surface layer lens on the element to focus its directivity pattern. Standard devices are typically housed in polymer catheter sheath whose material properties will have an imperfect focusing effect by refraction through the standard catheter body. By judicious choice of material, the catheter body can shift the effective focal depth shallower, but it will come at the expense of an ideal deep focus. It is not possible to focus individual lines at more than one depth with a standard single element, and the near field typically suffers.

In various embodiments, where an aperture is composed of an array of elements, a lens effect may be achieved by electronically delaying signals with respect to one another. One advantage in this embodiment is the ability to quickly adjust the focus specification. In one embodiment, this allows a multiple-element array system to dynamically adjust the receive focus during the reception phase so that all depths of the image are in focus for receive, which is called “dynamic focus.” Dynamic transmit focus is typically not possible within a single pulse-echo event even for arrays, because the focus profile is fixed at the time of emission. In one embodiment, a synthetic aperture method builds dynamic transmit focus in the context of an array-based system. For instance, a traditional but time consuming and computation intensive approach would be to excite each element of a multi-element array individually and acquire receive data for all elements either in parallel or by repetition of transmit and then form a beam for the image. For standard electronic circumferential multi-element phased array IVUS devices, electronic focusing can be achieved. But these standard multi-element arrays often need significantly complex electronics for the disposable catheter, and limitations of these electronics can reduce image quality in other ways, e.g. lower frame rate, resolution, etc. Dynamic focusing which is common with multi-element transducers may not be possible on transmit or receive for single element devices in certain embodiments. The systems and methods described herein are directed towards creating improved intravascular ultrasound imaging. More specifically, the systems and methods described herein produce an intravascular ultrasound image with enhanced and improved focusing across a range of depths, or distances from a rotating ultrasound transducer. In various embodiments, a lateral response of scan lines formed from individual passively focused pulse-echo events performed at various positions during the rotation of a single element transducer within an elongated jacket will be improved by a phase sensitive sum of several neighboring scan lines that have each been judiciously adjusted in amplitude and/or phase, along with optional time axis shifting dependent on their depth and relative angular position.

In one embodiment of a spectrally wideband IVUS imaging system, adjusting time of flight is used to align signals with respect to both the carrier frequency and excitation pulse envelope. In one embodiment, the signal processing implementation for improved focusing produces results similar to the delay, apodization, and coherent summation of a beamformer. However, unlike the application of a multi-element array beamformer, in various embodiments, the IVUS system with a single rotating transducer operations are applied to a sliding window of ultrasound lines, not as a reductive combination of element signals from within a multi-element array. Furthermore, these original lines are individual round-trip image lines, each from an individual pulse-echo event from a passive rotating device where no previous active focusing can have been applied. The methodology for computing the scaling and delay and/or phase control per line can vary due to specifics of the signal processing implementation. The apodization and delay/phase control will conform generally to linearizing the phase of the modified point spread function in azimuth. For example, in one embodiment, the conjugate of phase seen in the multiplicity of signals being combined in this operation for each point in the image, for the case where these signals correspond to a point scatter at the current point of interest, may be determined and applied.

In various embodiments, the effective focus for each individual scan line is improved via improved focusing by a phase sensitive combination of a plurality of neighboring scan line signals via properly adjusting amplitude, phase, and/or time axis adjustments. In one embodiment, the effective focus for each individual scan line is improved by a phase sensitive combination of a plurality of neighboring scan line signals via properly adjusting amplitude. In one embodiment, the effective focus for each individual scan line is improved by a phase sensitive combination of a plurality of neighboring scan line signals via properly adjusting phase. In one embodiment, the effective focus for each individual scan line is improved by a phase sensitive combination of a plurality of neighboring scan line signals via properly adjusting time axis adjustments. In various embodiments, 2-400 scan lines are used (e.g., 2, 6, 8, 10, 12, 14, 16, 18, 20, 24, 25, 30, 32, 35, 40, 50, 100, 150, 200, 250, 300, 350, 400 scan lines and values therein, and 2-16, 2-32, 2-64, 2-128, 2-200, 2-300, 2-400 scan lines and ranges therein).

In various embodiments, an improved imaging IVUS catheter deployed ultrasound device acquires frames of polar coordinate image lines. In one embodiment, fixed per-line focusing acquisition of image lines is acquired with a single element rotating transducer. In one embodiment, focus spatial resolution is improved with unique time/phase/amplitude adjustment of multiple acquisition lines followed by phase sensitive summation to generate each final improved display line.

As used herein, the term “proximal” may refer to a direction of the catheter that is typically pointed towards a user (e.g., the direction of the interface), whereas the term “distal” may refer to a direction of the catheter that is typically pointed away from the user (e.g., into the patient). As used herein, “composite frame line” may refer to a combination of two or more lines. In some embodiments, the imaging field may be defined as a polar coordinate system centered at a rotational transducer. As used herein azimuth shall be given its ordinary meaning and shall include an angle between an imaging line and a fixed axis extending from the center.

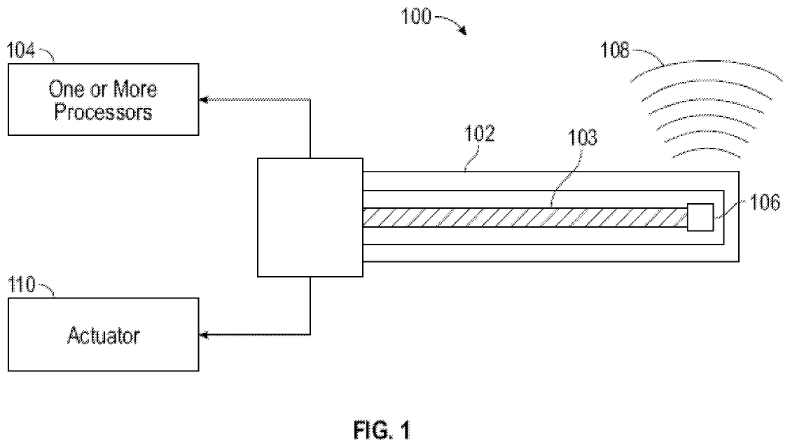

FIG. 1 illustrates a rotational intravascular ultrasound (IVUS) system 100 according to one embodiment. The intravascular ultrasound system 100 includes a catheter body 102. In some embodiments, the catheter body 102 may include one or more material layers to refract ultrasonic signals and/or backscatter signals in an intended manner.

In one embodiment, a rotational transducer 106 is disposed at the distal end of the catheter body 102 and generates a plurality of ultrasonic waves 108. In some embodiments, the rotational transducer 106 is oriented such that ultrasonic signals propagate mostly perpendicular to the catheter body 102. In some embodiments, the rotational transducer 106 is oriented such that ultrasonic signals propagate mostly perpendicular to an axis of the catheter body 102. In one embodiment, the rotational transducer 106 detects a plurality of backscatter signals. In some embodiments, the system 100 may include one generating transducer and one receiving transducer. In various embodiments, the rotational transducer 106 may be a single-element transducer or a multi-element array of transducers. In one embodiment, the rotational transducer 106 is only a single-element transducer (with no more than one transducer with a single element).

The system 100 may also include an actuator 110. The actuator 110 can comprise a component or system that is configured to cause relative motion (e.g., spinning motion, rotational motion) between two or more components, such as a driveshaft 103 (e.g., a motion between the rotational transducer 106 and the catheter body 102).

In one embodiment, the actuator 110 rotates the rotational transducer 106 within the body of the catheter 102 in an azimuthal direction. The rotational transducer 106 is mechanically connected to an actuator 110. For example, the rotational transducer 106 is connected to the actuator 110 via a driveshaft 103. The driveshaft 103 is disposed within the catheter body 102, and the rotational transducer 106 is disposed at the distal tip of the driveshaft 103. In some embodiments, the driveshaft 103 is disposed within a sheath. In various embodiments, a lumen of the catheter body 102 is filled with an acoustic coupling medium, such as an acoustic coupling fluid.

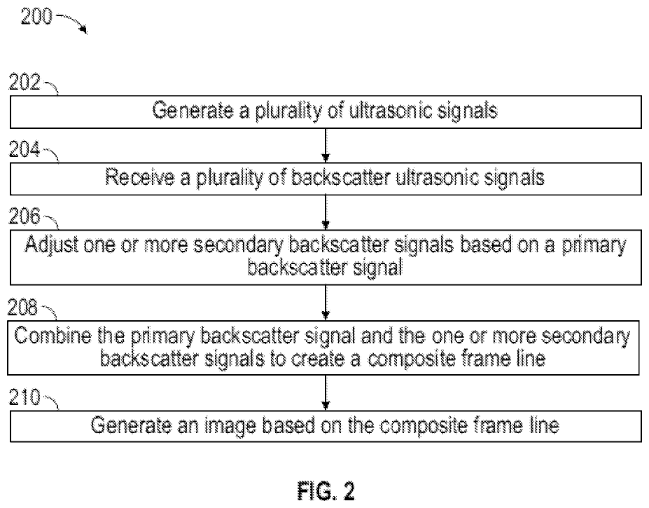

In various embodiments of employing the method, rotating or spinning a single ultrasound transducer at a rate between 2-100 rotations per second improves the clarity and sharpness of the imaging by 5-200% or more. In various embodiments, IVUS imaging resolution is improved 5-200% (e.g., 5, 10, 15, 20, 25, 30, 35, 40, 45, 50, 55, 60, 65, 70, 75, 80, 85, 90, 95, 100, 110, 120, 130, 140, 150, 160, 170, 180, 190, 200% and any values and ranges therein). In one embodiment, one, two or more processors 104 control the actuator 110 and the rotation of the rotational transducer 106 at the distal end of the catheter body 102. For example, the processors 104 may instruct the actuator 110 to rotate the rotational transducer 106 at a rate between 2-100 rotations per second (rps) (e.g., 2, 5, 10, 15, 20, 22, 24, 25, 26, 28, 29, 30, 31, 32, 33, 34, 35, 36, 37, 38, 39, 40, 41, 42, 43, 44, 45, 50, 55, 56, 57, 58, 59, 60, 61, 62, 63, 64, 65, 70, 75, 80, 85, 90, 95, 100 rps, 10-30, 10-50, 10-70, 10-90, 15-30, 20-30, 20-35, 20-40, 20-45, 20-50, 50-75, 75-100 and any ranges and values therein. In various embodiments, the rps is in a range of 30-60 rps, 20-40 rps, 25-65 rps, 15-45 rps (e.g., 20, 30, 40, 50, 60 rps). In some embodiments, the actuator 110 rotates the rotational transducer 106 at a fixed rate. In some embodiments, the actuator 110 rotates the rotational transducer 106 at one or more variable rates. The processors 104 may instruct the rotational transducer 106 to generate ultrasonic signals and detect backscatter signals generated by the tissue surrounding the catheter body 102 in response to the ultrasonic signals. In some embodiments, the processors 104 may cycle the rotational transducer 106 between generating ultrasonic signals and detecting backscatter signals. For example, the processors 104 may generate an electrical pulse that causes the rotational transducer to emit an ultrasonic waves 108. In various embodiments, ultrasonic pulse-echo images are formed from a plurality of pulse-echo events. A pulse echo event includes a transmission phase where a typically short pulse of acoustic energy is emitted followed by a reception phase where energy returning from echoes from the interrogated medium are sensed and processed. The recorded time of flight of a typically short high-bandwidth pulse provides the ability to discriminate the depth from which an echo originates. In one embodiment, this corresponds to the radial dimension of polar coordinate image data formation. The ability to discern the direction from which echo originates is governed by acoustic diffraction. This corresponds to the azimuthal dimension of polar coordinate data image formation and also the elevation or out of plane dimension. In one embodiment, the diffraction pattern is controlled principally by frequency content and aperture. The aperture is the surface used to create and sense the acoustic energy, such as that defined by the face of a single transducer element. The plurality of ultrasonic signals may include ultrasonic signals with varying frequencies and/or amplitudes. The rotational transducer 106 may detect a backscatter signal generated by the tissue surrounding the catheter body in response to the ultrasonic waves 108. The processors 104 may receive backscatter signals from the rotational transducer 106 and generate an ultrasound image as discussed in conjunction with FIG. 2. In some embodiments, the processors 104 may process the backscatter signals by filtering, receive beamforming, adjusting, and/or transforming the backscatter signals. The processors 104 may execute on an imaging software platform. The imaging software platform may provide a user interface for the control of the system 100. The imaging software platform may provide a multiple client interface and an image data platform. There may be one, two or more processors according to alternate embodiments. In several embodiments, generated images are displayed, for example on a monitor, screen, head-mounted display, etc.

In one embodiment, FIG. 2 depicts a process 200 for creating an intravascular ultrasound image performed by one or more processors, such as the one or more processors 104. As discussed above, the one or more processors 104 may control the elements of an intravascular ultrasound catheter system, such as the rotational transducer 106 and/or the actuator 110 of the system 100.

In one embodiment, the process 200 begins with step 202 where the one or more processors 104 instruct the rotational transducer 106 to generate one or more ultrasonic signals. The one or more processors 104 may instruct the actuator 110 to rotate the rotational transducer 106 within the catheter body. During rotation the one or more processors 104 may instruct the rotational transducer 106 to generate one or more ultrasonic signals along a plurality of lines. As indicated above, the tissue surrounding an intravascular ultrasound system may be divided into a plurality of lines disposed at various angles across the imaging plane. Along each line, imaging targets are located at various depths or radii from the rotating transducer.

In one embodiment, each ultrasonic signal includes a single ultrasonic signal with a particular amplitude as a function of time, depth, radii, alone or in combination. Each ultrasonic signal may also include a plurality of ultrasonic components in other embodiments. In some embodiments, the one or more processors 104 may generate the one or more ultrasonic signals based on a target depth. The processors 104 may focus the one or more ultrasonic signals by generating ultrasonic signals with a particular frequency, a particular amplitude, and/or a particular sequence and/or generating the ultrasonic signals at a particular rate. In some embodiments, the one or more ultrasonic signals may include two or more frequencies to provide for a dual frequency pulse excitation.

The process 200 may move to step 204 where the processors 104 instruct the rotational transducer 106 to detect one or more backscatter signals. For each line, the one or more processors 104 may detect a corresponding backscatter signal generated by the surrounding tissue in response to one or more ultrasonic signals. In some embodiments, the processors 104 may store the backscatter signals in a memory. For example, as the transducer 106 is rotated, the processors 104 may instruct the rotational transducer to generate a plurality of ultrasonic signals for a plurality of lines and collect a corresponding plurality of backscatter signals for each of the plurality of lines. The plurality of backscatter signals is divided by the one or more processors 104 into a primary backscatter signal and one or more secondary backscatter signals. In some embodiments, adjacent lines may not include an immediately adjacent line to the particular line but may include lines based on criteria such as the characteristics of the neighboring line's ultrasonic signal.

The process 200 may move to step 206 where the one or more processors 104 adjust(s) the one or more secondary backscatter signals. The processors 104 may adjust a time, a phase, and/or an amplitude of the secondary backscatter signals. The one or more secondary backscatter signals are adjusted based on the relationship to the primary backscatter signal for the case where a point target is positioned at the point of interest, using a delay function and/or an apodization protocol. The delay function and apodization protocol may vary based on a target depth. In some embodiments, the one or more processors 104 may adjust both the primary backscatter signal and the secondary backscatter signal(s). In various embodiments, signal processing for focus improvement comprises using delay and/or apodization to a sliding window of acquired ultrasound lines rather than a standard plurality of individual element signals. In various embodiments, the term apodization includes for example weighting in proportion to the received signal(s) when a point target is imaged such as in matched filtering, employing a whitening apodization such as a Weiner filter, or another strategy to optimize image characteristics (e.g., such as sharpness and/or clarity).

In one embodiment, rotational transducer 106 can generate wideband ultrasonic signals, the one or more processors 104 may adjust a time of flight of the backscatter signals. In some embodiments, the rotational transducer 106 is a narrowband or a continuous wave (CW) imaging system, and the one or more processors 104 may adjust the secondary backscatter signals using a complex analytic filter and a convolution protocol.

In various embodiments of a narrowband or continuous wave (CW) imaging system, signal processing for focus improvement reduces blurriness with a complex analytic filter, where the ideal phase provided for the filter is the complex conjugate of the imager point spread function at each given depth. In one embodiment, signal processing for focus improvement is applied to a wideband imaging system.

In some embodiments, control parameters of the focus improvement are implemented in a range dependent way so that the focus improvement is enhanced and/or maximized for more, or all depths. In various embodiments, adjustments vary as function of depth.

The delay function may include one or more control parameters that vary in a depth-dependent way.

In some embodiments, the process 200 may proceed to step 208 where the primary backscatter signal and the one or more secondary backscatter signals are combined to form a composite frame line. The primary backscatter signal and the one or more secondary backscatter signals is summed in a phase sensitive manner. In some embodiments, the primary backscatter signal and the one or more secondary backscatter signals are summed using weighted values.

In some embodiments, the one or more processors 104 may repeat steps 206 and 208 for a plurality of target depths. The delay function may include one or more control parameters that vary in a depth-dependent way. For example, the one or more processors 104 may adjust the secondary backscatter signals using a first delay function and/or a first apodization protocol that correspond with a first target depth and combine the adjusted secondary backscatter signals with the primary backscatter signal to form a first composite frame line. Then, the processors 104 may adjust the secondary backscatter signals using a second delay function and/or a second apodization protocol that correspond with a second target depth and combine the adjusted secondary backscatter signals with the primary backscatter signal to form a second composite frame line. The first and second composite frame lines are combined to create the ultrasound image. One, two or more processors 104 may be used.

The process 200 may proceed to step 210 where the one or more processors 104 generate an ultrasound image based on the composite frame line. The ultrasound image is continuously updated as the one or more processors repeat steps 204-210 for subsequent lines. For example, the ultrasound image is updated after each composite frame line is created.

Advantageously, the system 100 and the process 200 provide a high frame rate ultrasound image with increased azimuthal focus in the near-field and far-field. In one embodiment, signal processing produces a high frame rate imaging within a computationally capable framework such as graphics processing unit (GPU) accelerated software-based imaging pipeline. By providing an improved image the diagnoses of vascular diseases, such as atherosclerosis, is made more accurately and reliably. More specifically, improved near-field focus allows physicians to assess the diameter of the lumen and extent of blockage caused by atherosclerotic plaques. Vascular treatments, such as the placement of stents, may also be improved. The systems and methods described above may enable physicians to verify a stent has been correctly placed, allowing patients to avoid complications and subsequent removal of the stent.

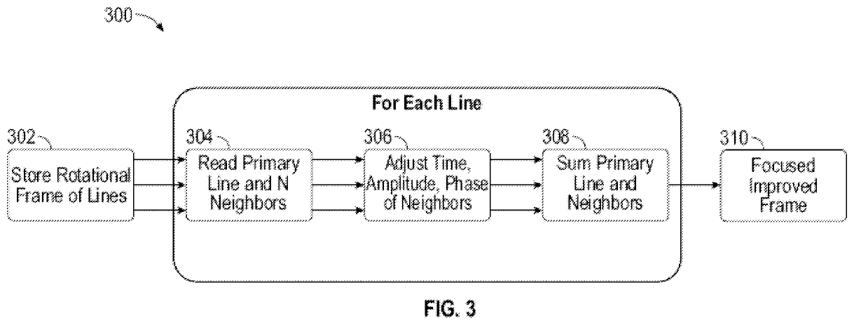

FIG. 3 illustrates an embodiment of a process 300 for processing an intravascular ultrasound image. In one embodiment, the process 300 begins at step 302 where one or more processors collect and store rotational frames of lines.

For each frame line, the one or more processors execute steps 304, 306, and 308. At step 304, the one or more processors read a primary line and an N number of neighboring lines.

In some embodiments, the one or more processors move to step 306 and adjust the time, amplitude, and/or phase of the neighboring lines.

In one embodiment, the process 300 moves to step 308 where the one or more processors sum the primary line and the N number of neighboring lines to create a composite frame line.

In one embodiment, the process 300 moves to step 310 where the one or more processors create a focused, improved frame based on the composite frame line.

In some embodiments a spectrally wideband imaging system is employed, and time of flight adjustment is necessary to align signals with respect to both the carrier frequency and excitation pulse envelope. The one or more processors may leverage a delay function and an apodization protocol to a sliding window of acquired ultrasound lines. In some embodiments, a narrowband or continuous wave (CW) imaging system is employed, and the one or more processors may leverage convolution with a complex analytic or lateral filter to create the focused, improved ultrasound image. The complex analytic filter may use an ideal phase profile as a filter. The ideal phase profile is the complex conjugate of the imager point spread function at each given depth. In some embodiments, the control parameters of the focus improvement are implemented in a range dependent way so that the focus improvement is maximized for all depths. The adjustments may vary as a function of depth. In some embodiments the ideal control parameters calculated for the case of a CW imaging system may be deployed in the case of a wideband imaging system as an approximation or simplification.

In one embodiment, control parameters of the IVUS focus improvement will be implemented in a range dependent way so that the focus improvement may be maximized for all depths and distances from the catheter body. In one embodiment, adjustments are varied as a function of depth/distance.

In several embodiments, IVUS imaging improvements can be achieved in the case where catheter body material is affecting the passive natural focus adversely. For instance, if the catheter body has a detrimental effect on focusing in the far field, or if it even has a defocusing effect at several depths, this technique will improve performance depending on the degree of original defocusing. In one embodiment, the opportunity for improvement is greater when the original focus is less ideal and there will be no (or minimal) advantage at a depth that is already perfectly focused. In one embodiment, an adjustment of amplitude is used to modulate the effect of the IVUS imaging method so that it can be disabled at a depth that is originally focused.

In one embodiment, improvements are achieved in the case where the directivity function is degraded by the continuous rotation of the device leading to misalignment of transmit and receive directivities.

In one embodiment, potential downsides of traditional processing, such as parasitic smoothing and artifact production due to target motion in the angular direction are reduced or eliminated.

In one embodiment, the individual signals for the rotating transducer that are available for combination do not represent different individual sub-apertures. In one embodiment, the individual signals for the rotating transducer that are available for combination do not represent synthetic aperture imaging by acquiring different sub-apertures per pulse-echo event. In one embodiment, the signals are from the same single transducer and they do not represent same receive aperture specifications with different transmit profiles according to certain embodiments of transmit focus improvement. In one embodiment, the signals are from the same single transducer and they do not represent receive apertures with different transmit profiles from a phased array device. In one embodiment, the aperture implemented by a single transducer is not different between transmit and receive, and produces only a line of round-trip focused data per pulse-echo event. It may be rotating between each pulse echo event to affect the direction to which an ultrasound line corresponds. In one embodiment, both transmit and receive apertures are complete and not subdivided or different with respect to the single signal produced by each pulse echo event. In one embodiment, both transmit and receive apertures are statically focused, unfocussed, or imperfectly focused (to the extent that the energy contribution from each point along the aperture is not substantially equal phase). In various embodiments, a natural focus can be unfocussed or imperfectly focused. In one embodiment, a static focus can be achieved with a lens. In one embodiment, an annular cross-section catheter body approximates a lens. In one embodiment, an annular cross-section catheter body is not approximated by a lens.

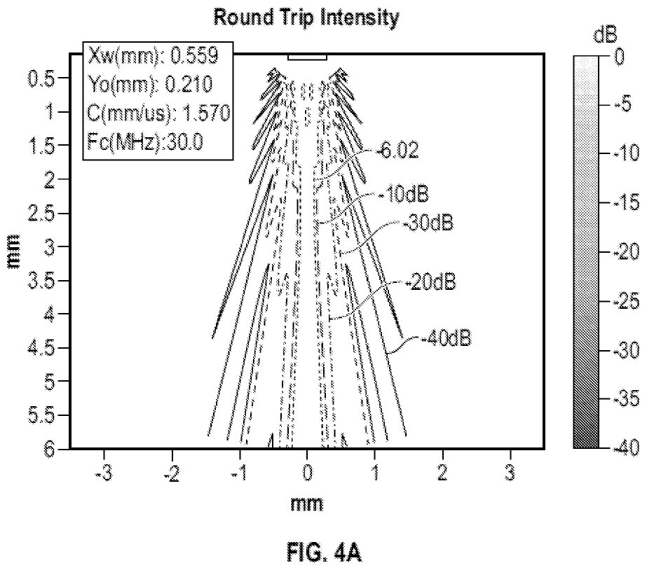

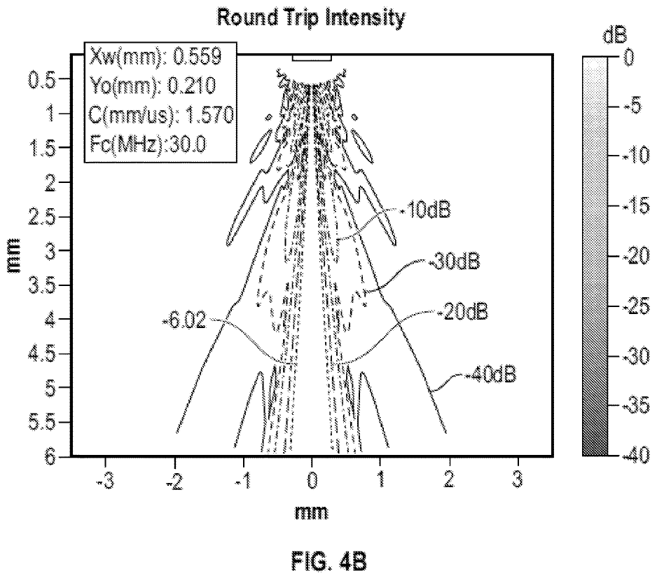

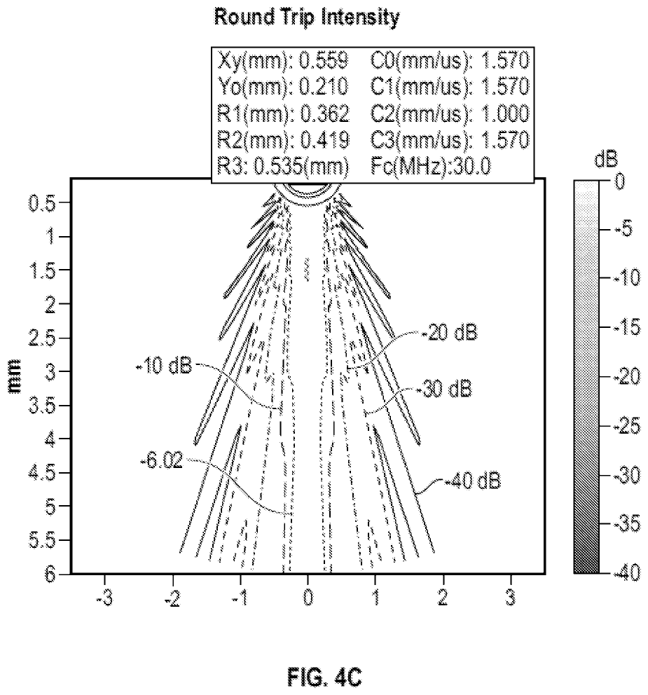

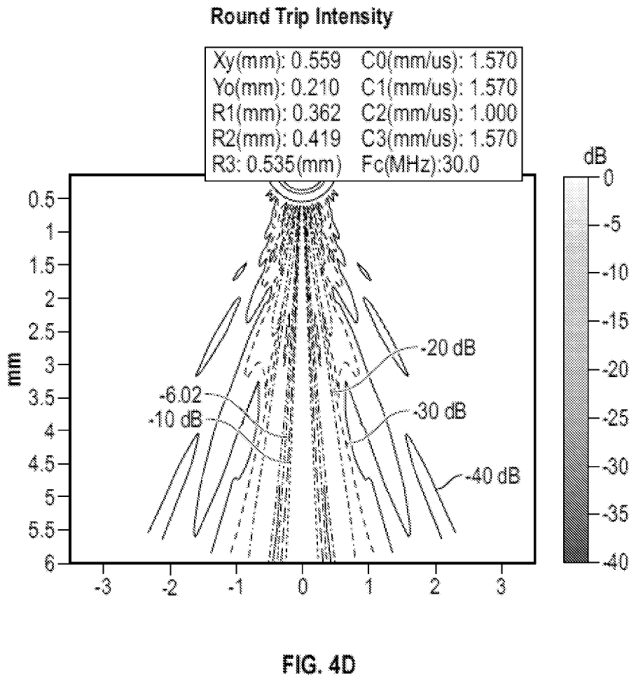

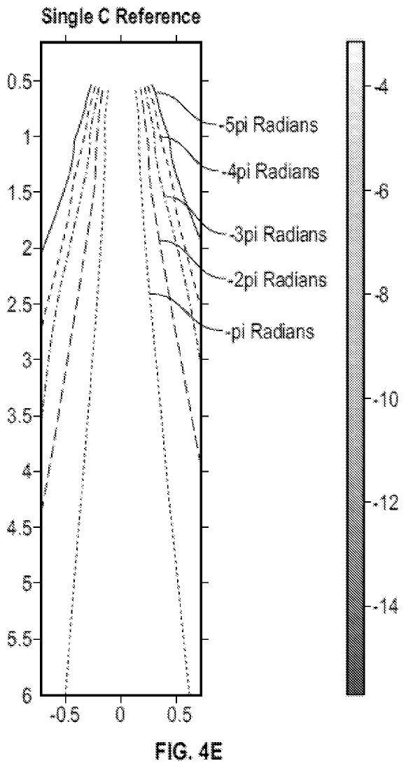

FIG. 4A-4D are a series of contour plots illustrating the angular sensitivity of continuous wave (CW) system in certain simulations according to several embodiments. The plots represent the shapes, or profiles, of acoustic beam intensity, and are good approximations for non-CW imaging where broadband pulses are deployed according to several embodiments.

FIG. 4A illustrates a simulation of the angular sensitivity profile as a function of imaging depth for a purely passive single focus rotating transducer with no additional refracting catheter jacket material, along an x-axis in mm, y-axis in mm. FIG. 4A illustrates an embodiment of the simulated round trip intensity in the imaging plane of an unfocussed (“focus at infinity”) single element 30 MHz ultrasound transducer without focus improvement. FIG. 4A illustrates round-trip intensity for a single medium passively focused with contours at −6.02 dB near the middle, −10 dB outward, −20 dB outward, −30 dB outward, and −40 dB on the outside (left and right) with a width X_{w} of 0.559 mm, a depth of y₀ 0.210 mm, speed of sound c of 1.570 mm/μs at a frequency of 30.0 MHz.

FIG. 4B illustrates a simulation of transmit focusing improvement of the sensitivity profile in the near field. In one embodiment, improved focusing creating a more narrow beam width shown in FIG. 4B compared to FIG. 4A is significant in the near field, however at the natural focus and beyond there is minimal to no improvement as the original case is already focused for these depths. FIG. 4B illustrates an embodiment of simulated round trip Intensity in the imaging plane of an unfocussed (“focus at infinity”) single element 30 MHz ultrasound transducer with focus improvement according to embodiments disclosed herein. FIG. 4B illustrates round-trip intensity for a single imaged medium with improved focus with contours at −6.02 dB near the middle, −10 dB outward, −20 dB outward, −30 dB outward, and −40 dB on the outside (left and right) with a width X_{w} of 0.559 mm, a depth of y₀ 0.210 mm, speed of sound c of 1.570 mm/μs at a frequency of 30.0 MHz. In one embodiment with the −6 dB azimuthal beamwidth provides a proxy for azimuthal detail resolution, in this case of no additional refractive material achieves a minimum of approximately 0.20 mm at 2 mm imaging depth in simulation. For example, in one embodiment with improved focusing improvement the resolution is reduced to 0.15 mm, a 25% improvement. Resolution improvement is greater in the near field, for example reducing from 0.40 mm to 0.06 mm at 1 mm depth, an 85% improvement. The improvement reduces in the far field, reaching parity at approximately 5.5 mm depth.

FIG. 4C illustrates a simulated round-trip normalized (each range) amplitude for imaging where the single focus has been degraded by an unfavorable choice of speed of sound in a catheter body. FIG. 4C illustrates an embodiment of simulated round trip intensity in the imaging plane of single element 30 MHz ultrasound transducer surrounded by catheter body of poor material property without focus improvement. FIG. 4C illustrates contours at −6.02 dB near the middle, −10 dB outward, −20 dB outward, −30dB outward, and −40 dB on the outside (left and right) with a width X_{w} of 0.559 mm, a depth of y₀ 0.210 mm, r1 at 0.362 mm, r2 at 0.419 mm, r3 at 0.535 mm, speed of sound c0 of 1.570 mm/μs, speed of sound c1 of 1.570 mm/μs, speed of sound c2 of 1.000 mm/μs, and speed of sound c3 of 1.570 mm/μs at a frequency of 30.0 MHz. This represents an embodiment of a transducer surrounded by an acoustic coupling fluid, and a material for the catheter body.

FIG. 4D illustrates a simulated round-trip intensity of the improved focusing method that recovers unfavorable angular resolution providing a significant benefit in image quality. FIG. 4C illustrates an embodiment of simulated round trip intensity in the imaging plane of single element 30 MHz ultrasound transducer surrounded by catheter body of poor material property with focus improvement according to embodiments disclosed herein. FIG. 4D illustrates contours at −6.02 dB near the middle, −10 dB outward, −20 dB outward, −30 dB outward, and −40 dB on the outside (left and right) with a width X_{w} of 0.559 mm, a depth of y₀ 0.210 mm, r1 at 0.362 mm, r2 at 0.419 mm, r3 at 0.535 mm, speed of sound c0 of 1.570 mm/μs, speed of sound c1 of 1.570 mm/μs, speed of sound c2 of 1.000 mm/μs, and speed of sound c3 of 1.570 mm/μs at a frequency of 30.0 MHz. In this example with an unfavorable sound speed in the catheter body, the simulated azimuthal −6 dB beamwidth at 2 mm depth is 0.46 mm without focusing improvement and 0.13 mm with focus improvement, a 71% improvement. At 1 mm the beamwidth reduces from 0.52 mm to 0.06 mm, or 88%. At 6 mm depth the beamwidth reduces from 0.52 to 0.48, or 8%.

In various embodiments, focus quality is measured via comparison of the spatial decorrelation length of speckle to the width of small specular points in an image. In one embodiment with a focused imaging system, these measures will be similar, and as the focus is degraded, the width of the specular point targets increases while the length of the speckle decorrelation does not. In one embodiment, phase sensitive summation across lines is detected by observing interference patterns in an image or images when injecting gated pulses from a signal generator.

In various embodiments, a method for processing ultrasound data from a rotating transducer situated within an elongated member includes acquiring an original plurality of image lines, where each of the original plurality of image lines is acquired by a distinct pulse-echo emission and reception, and each of the original plurality of image lines corresponds to substantially one angular direction according to the physics of diffraction.

In various embodiments, a method for processing ultrasound data from a rotating transducer situated within an elongated member includes processing the original plurality of lines to produce a modified plurality of lines with improved angular directivity characteristics by a phase sensitive summation of several of the original plurality of image lines to produce each of a modified plurality of image lines, and a modification by phase rotation and/or time shifting with or without additionally scaling amplitude prior to the phase sensitive summation, where the modification by phase rotation, time shifting, or both phase rotation and time shifting with or without additionally scaling amplitude is constructed so that the modified plurality of image lines will exhibit a more linear phase than the original plurality of lines about a direction corresponding to the location of a point target when imaged.

In one embodiment, the amplitude scaling values may be taken as the simulation intensity without correction, such has been used in some of the simulation examples here. For example, the amplitudes illustrated in FIGS. 4A and 4C for the continuous wave cases modeling a single propagation media and unfavorable jacket material choice respectively. In one embodiment, the amplitude scaling may be taken from an experimental measurement of intensity without correction. In one embodiment, the amplitude scaling may be informed by a heuristic model approximating one of the above. In some embodiments, a number of different strategies may be employed for the amplitude weighting such as may be designed to accomplish various tradeoffs between main peak of the resulting intensity function and sidelobes, for instance. Note that if one were to truncate the contributors from neighboring original lines based on a threshold value of weighting fewer lines would contribute at depths which are already previously focused, as has been discussed already.

In one embodiment, a phase adjustment applied prior to phase sensitive summation may be calculated as the complex conjugate of the continuous wave simulation of phase from the reflection off of a point target at the current point of interest. In one embodiment, this phase may be used directly and without time adjustment as would be exactly appropriate for continuous wave imaging and approximately appropriate for the case of narrowband imaging. In one embodiment, a time shift may be inferred from the phase described previously by unwrapping and scaling, which could be applied for instance as a time adjustment in the case of wideband imaging. In one embodiment, a time adjustment for wideband imaging may be computed based on simulated wavefront position and slope through refracting material of the catheter body versus the ideal wavefront to achieve focus at the point of interest.

In one embodiment, the computed phase adjustment as a function position relative to a central line is shown in FIG. 4E with respect to the contour plot of FIG. 4D for the case of a single propagation medium. The geometric conditions for this simulation in FIG. 4D are identical to those of FIGS. 4A and 4B. The contours levels shown are −pi, −2 pi, −3 pi, −4 pi, −5 pi radians for the sequence of lines near the center (−pi) and moving outward toward the sides in the sequence −2 pi, −3 pi, −4 pi, and then −5 pi radians.

In one embodiment, the modification prior to summation is a function of imaging depth.

In one embodiment, the improvement of focusing is achieved for point targets imaged at multiple depths in the image.

In one embodiment, the improvement of focusing improves spatial resolution by narrowing the main peak of the response from the point target in the lateral dimension.

In one embodiment, the improvement of focusing improves clutter rejection by lowering the response from the point target away from the main peak in the lateral dimension.

In various embodiments, the improvement of focusing is applied to interleaved line acquisition. In one embodiment, near and far line segments are quickly acquired as an inner loop so the operation is applied across like segments at different angles. In one embodiment, odd lines are combined to generate new odd lines and/or even lines are combined to generate new even lines.

In various embodiments, a method for processing ultrasound data from a rotating transducer includes: acquiring an original plurality of image lines from a rotating ultrasound transducer, wherein the rotating ultrasound transducer is attached to a driveshaft positioned in a lumen of a flexible, elongate member, wherein each of the original plurality of image lines is acquired by a pulse-echo emission and a pulse-echo reception, and wherein each of the original plurality of image lines corresponds to an angular direction where the degree to which each angle can be discriminated, called the angular directivity, is governed by physics of diffraction as it applied to the physical construction of the device. In one embodiment, the method further includes processing the original plurality of image lines to produce a modified plurality of image lines with improved angular directivity characteristics. In various embodiments, the modified plurality of image lines is produced via a modification by one or more of: a phase rotation, a time shift, and a phase sensitive summation of at least two of the original plurality of image lines to produce each of the modified plurality of image lines. In one embodiment, a modification via phase rotation and/or time shift is configured to produce the modified plurality of image lines with a more linear phase than the original plurality of image about a direction corresponding to a location of one or more targets when imaged. In various embodiments, the modified plurality of images lines exhibits less than ¼ cycle of phase variation from linear within the extent of a main-lobe width defined by 10 dB down from peak criteria when imaging an isolated point scatter, whereas the original plurality of lines does not.

In one embodiment, each of the original plurality of image lines corresponds to a single angular direction with a polar coordinate. Phase rotation and/or time shifting may further include scaling amplitude. Amplitude scaling may be done prior to the phase sensitive summation of the at least two of the original plurality of image lines. Phase rotation and/or the time shift as well as the amplitude scaling may be a function of an imaging depth. In one embodiment, an improvement of image focusing is achieved for the target(s) imaged at one or more depths in an image. In one embodiment, spatial resolution is improved by narrowing a main peak of the pulse-echo reception from the one or more targets in a lateral dimension. In one embodiment, clutter rejection is improved by lowering the response from the one or more targets away from a main peak in a lateral dimension. In various embodiments, the main peak is narrowed by 5-100% (e.g., 5%, 10%, 15%, 20%, 25%, 30%, 35%, 40%, 45%, 50%, 60%, 70%, 80%, 90%, 99%, 100% or more, and overlapping ranges therein, such as 5-99%, 5-90%, 5-80%, 5-75%, 5-70%, 5-60%, 5-50%, 5-25%, 5-10%).

In various embodiments, a method for improved focusing of an intravascular ultrasound catheter system includes generating a plurality of ultrasonic signals, receiving a plurality of backscatter ultrasonic signals, wherein the plurality of backscatter ultrasonic signals comprises a primary backscatter signal and one or more secondary backscatter signals, adjusting the one or more secondary backscatter signals based on the relationship of to the primary backscatter signal for the case of a point target at the point of interest, or a simulation of the primary backscatter signal expected from the case of an isolated point target at the point of interest, combining the primary backscatter signal and the one or more secondary backscatter signals to form a composite frame line; and generating an image based on the composite frame line.

Artificial Intelligence: In several embodiments, the systems described herein, including for example the advanced intravascular ultrasound platform, leverage AI to enable image interpretation, enhance total-system capabilities, and streamline workflows to maximize the clinical value. In some embodiments, advantageously, physicians will not need to integrate (e.g., cognitively integrate) imaging data spatially and temporally to fully interpret the clinical condition. Instead, systems according to several embodiments described herein can leverage the power of AI with generational advancements to go beyond single image interpretation. In several embodiments, the AI-powered engine, for example, may include a workstation that enhances image interpretation with a simplified workflow improving overall useability. Machine learning is used in several embodiments. In one embodiment, the AI-ready processing power is designed to support real time and on-demand image interpretation. The AI powered workstation can provide high end processing and an AI engine for advanced signal and image processing. In various embodiments, the native image data capture provides for superior image interpretation (e.g., border detection, identification and measurement of vessel size, vessel disease, dissection, plaque morphology, etc.). In several embodiments, the systems described herein provide simplified measurement via automated border detection (e.g., AI algorithms automatically identify borders of a lumen, vessel, tissue, lesion, plaque, etc.). In several embodiments, the system provides simplified measurement via semi-automated border detection (e.g., the user can manually adjust or modify automated AI algorithms that identify borders of a lumen, vessel, tissue, lesion, plaque, etc. with the border selection reconfigured based on user modifications). In one embodiment, AI plaque identification utilizes AI algorithms to automatically classify and identify types of plaque within the imaged area to provide user guidance on treatment options (e.g., using color coding, icons or text overlays can be used to indicate what type of condition, such as plaque, may be present for the selected image). In several embodiments, the data driven platform is designed to collect data, simplify image interpretation, with AI processing power to support real time and on-demand image interpretation and reduce user cognitive load to help (i) identify lumen size, (ii) visualize dissections, (iii) characterize disease morphology, (iv) locate and quantify stenosis, and/or (v) identify true lumen. In some embodiments, image interpretation is used to identify thrombus, thrombosis, clots, embolisms, plaque, calcium, tissue health, stent or balloon apposition, and/or stent or balloon “health” or condition. Image interpretation may involve imaging to evaluate quality and/or position of placement of an existing stent. Image interpretation can involve identifying position relative to lumen walls, determine level of and/or quality of tissue grown into and around the stent or balloon. In one embodiment, for example with a bioresorbable stent, image interpretation can involve (i) evaluating the amount of dissolving of the stent, (ii) determining if the dissolving of the stent is in accordance with expected decay patterns (e.g., determining whether the level of decay on one side of the stent similar to the other side of the stent, and if not, that may indicate a problem with stent placement, or if the stent is dissolving more rapidly than expected that could indicate the stent will not provide the tissue with the expected structural support).

The technology described herein is used in several embodiments with flush-less, encapsulated seal IVUS catheters to reduce air bubbles and eliminate need to flush acoustic coupling fluid including the technology described in U.S. Ser. No. 63/459,312 entitled Systems and Methods for Flush-Less Intravascular Ultrasound Catheter and U.S. Ser. No. 63/546,091 entitled Systems and Methods for Flush-Less Intravascular Ultrasound Catheter (and the PCT application claiming priority thereto and filed Apr. 11, 2024); manually assisted pullback for spatial alignment measurements, voice control, position sensors (e.g., encoder) including the technology described in U.S. Ser. No. 63/531,266 entitled Systems and Methods of Manually Assisted Pullback for Spatial Alignment Lengthwise Measurements in Intravascular Ultrasound Imaging; and ultrasound imaging systems and components, voice control, and artificial intelligence algorithms including the technology described in U.S. Ser. No. 63/546,058 entitled Systems and Methods for Intravascular Ultrasound, all herein incorporated by reference in their entirety into the disclosure.

Changes and modifications in the embodiments described herein can be carried out without departing from the principles of the present disclosure. Each of the disclosed aspects and examples of the present disclosure may be considered individually or in combination with other aspects, examples, and variations of the disclosure. In addition, unless otherwise specified, none of the steps of the methods of the present disclosure are confined to any particular order of performance.

While the methods and devices described herein may be susceptible to various modifications and alternative forms, specific examples thereof have been shown in the drawings and are herein described in detail. Embodiments are not to be limited to the particular forms or methods disclosed, but rather intended is to cover modifications, equivalents, and alternatives falling within the spirit and scope of the various examples and embodiments described herein and/or in the appended claims. Further, the disclosure herein of any particular feature, aspect, method, property, characteristic, quality, attribute, element, or the like in connection with an example can be used in all other examples set forth herein. Any methods disclosed herein need not be performed in the order recited. The use of sequential, or time-ordered language, such as “then,” “next,” “after,” “subsequently,” and the like, unless specifically stated otherwise, or otherwise understood within the context as used, is generally intended to facilitate the flow of the text and is not intended to limit the sequence of operations performed. Thus, some examples may be performed using the sequence of operations described herein, while other examples may be performed following a different sequence of operations.

Conditional language used herein, such as, among others, “can,” “might,” “may,” “e.g.,” and the like, unless specifically stated otherwise, or otherwise understood within the context as used, is generally intended to convey that some examples include, while other examples do not include, certain features, elements, and/or states. Thus, such conditional language is not generally intended to imply that features, elements, blocks, and/or states are in any way required for one or more examples or that one or more examples necessarily include logic for deciding, with or without author input or prompting, whether these features, elements and/or states are included or are to be performed in any particular example. Where devices or methods “comprise” or “include” (the two being interchangeable) certain features or steps, such devices or methods may also “consist essentially of” such features or steps if identified as such in the claims. Where devices or methods “comprise” or “include” (the two being interchangeable) certain features or steps, such devices or methods may also “consist” of such features or steps if identified as such in the claims.

The methods disclosed herein may include certain actions taken by a practitioner; however, the methods can also include any user or third-party instruction of those actions, either expressly or by implication. For example, actions such as “positioning a device” include “instructing positioning of a device.”

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonable under the circumstances, for example ±5%, +10%, +15%, etc.). For example, “about 4 inches” includes “4 inches.” Phrases preceded by a term such as “substantially” include the recited phrase and should be interpreted based on the circumstances (e.g., as much as reasonably possible under the circumstances). For example, “substantially linear” includes “linear.” Unless stated otherwise, all measurements are at standard conditions including temperature and pressure. The phrase “at least one of” is intended to require at least one item from the subsequent listing, not one type of each item from each item in the subsequent listing. For example, “at least one of A, B, and C” can include A; B; C; A and B; A and C; B and C; or A, B, and C.

The various logics, logical blocks, modules, circuits and algorithm steps described in connection with the implementations disclosed herein can be implemented as electronic hardware, computer software, or combinations thereof. The hardware and data processing apparatus used to implement the various illustrative logics, logical blocks, modules and circuits described in connection with the aspects disclosed herein may be implemented or performed with a general-purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, or any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some implementations, particular steps and methods may be performed by circuitry that is specific to a given function. The functions described may be implemented in hardware, digital electronic circuitry, computer software, firmware, including the structures disclosed in this specification and their structural equivalents thereof, or in combinations thereof. Implementations of various embodiments can be implemented as one or more computer programs, such as one or more modules of computer program instructions, encoded on a computer storage media for execution by, or to control the operation of, data processing apparatus. If implemented in software, the functions may be stored on or transmitted over as one or more instructions or code on a computer-readable medium. The steps of a method or algorithm disclosed herein may be implemented in a processor-executable software module which may reside on a tangible, non-transitory computer-readable medium. Computer-readable media includes both computer storage media and communication media including any medium that can be enabled to transfer a computer program from one place to another. A storage media may be any available media that may be accessed by a computer. The operations of a method or algorithm may reside as one or any combination or set of codes and instructions on a machine readable medium and computer-readable medium, which may be incorporated into a computer program product.

1.-77. (canceled)

78. A system for processing ultrasound data from a rotating single element ultrasound transducer, the system comprising:
an intravascular ultrasound (IVUS) catheter comprising:
a flexible, elongate member comprising a lumen; and
a rotating single element ultrasound transducer attached to a driveshaft positioned in the lumen; and
a processor configured to be placed in electronic communication with the rotating single element ultrasound transducer, the processor configured to:
acquire an original plurality of image lines from the rotating single element ultrasound transducer,
wherein each of the original plurality of image lines is acquired by a pulse-echo emission and a pulse-echo reception in a single plane while the rotating single element ultrasound transducer is spinning about a length axis of the driveshaft, and
wherein each of the original plurality of image lines corresponds to an angular directivity within the single plane; and
process the original plurality of image lines to produce a modified plurality of image lines within the single plane with modified angular directivity characteristics via:
a modification by a phase rotation, and
a phase sensitive summation of at least two of the original plurality of image lines to produce each of the modified plurality of image lines,
wherein the modification by the phase rotation is configured to produce the modified plurality of image lines with a more linear phase than the original plurality of image lines about a direction corresponding to a location of one or more isolated point targets when imaged.

79. The system of claim 78, wherein:
the modification is a function of an imaging depth,
processing the original plurality of image lines comprises determining the one or more isolated point targets within the original plurality of image lines, and
the one or more isolated point targets are focused at one or more depths in an image.

80. The system of claim 78, wherein each of the original plurality of image lines corresponds to the angular directivity with a polar coordinate.

81. The system of claim 78, wherein the modification further comprises a time shift.

82. The system of claim 78, wherein the processor is configured to process the original plurality of image lines by determining the one or more isolated point targets within the original plurality of image lines.

83. The system of claim 78, wherein the one or more isolated point targets are focused at one or more depths in an image.

84. The system of claim 78, further comprising narrowing a main peak of the pulse-echo reception from the one or more isolated point targets for a spatial resolution in a lateral dimension.

85. The system of claim 78, wherein clutter rejection is reduced by lowering a response from the one or more isolated point targets away from a main peak in a lateral dimension.

86. The system of claim 78, wherein the phase sensitive summation comprises a phase sensitive vector summation of the at least two of the original plurality of image lines.

87. The system of claim 78, wherein the original plurality of image lines comprises between 2 scan lines and 400 scan lines.

88. The system of claim 78, wherein the original plurality of image lines are acquired at an imaging frequency between 1 megahertz (MHz) to 90 MHz.

89. The system of claim 78, wherein the system is configured for use in a peripheral vessel.

90. The system of claim 78, wherein the system is configured for use in a renal or coronary vessel.

91. The system of claim 78, wherein the system is configured for use in connection with angiography, a neurovascular procedure, or intravascular lithotripsy.

92. A method for processing ultrasound data from a rotating single element ultrasound transducer of an intravascular ultrasound (“IVUS”) catheter, the method comprising:
acquiring an original plurality of image lines from the rotating single element ultrasound transducer,
wherein the rotating single element ultrasound transducer is attached to a driveshaft positioned in a lumen of a flexible, elongate member of the IVUS catheter,
wherein each of the original plurality of image lines is acquired by a pulse-echo emission and a pulse-echo reception in a single plane while the rotating single element ultrasound transducer is spinning about a length axis of the driveshaft, and
wherein each of the original plurality of image lines corresponds to an angular directivity within the single plane,
wherein the angular directivity comprises a characteristic that is governed by diffraction; and
processing the original plurality of image lines to produce a modified plurality of image lines within the single plane with modified angular directivity characteristics via:
a modification by one or more of: scaling of an amplitude, a phase rotation, and a time shift, and
a phase sensitive summation of at least two of the original plurality of image lines to produce each of the modified plurality of image lines,
wherein the modification by one or more of: the scaling of the amplitude, the phase rotation, and the time shift is configured to produce the modified plurality of image lines with a more linear phase than the original plurality of image lines about a direction corresponding to a location of one or more isolated point targets when imaged.

93. The method of claim 92, wherein:
the modification comprises the phase rotation,
the modification is a function of an imaging depth,
processing the original plurality of image lines comprises determining the one or more point targets within the original plurality of image lines, and
the one or more isolated point targets are focused at one or more depths in an image.

94. The method of claim 92, wherein the modification further comprises at least one of the time shift or the scaling of the amplitude.

95. The method of claim 92, wherein processing the original plurality of image lines comprises determining the one or more isolated point targets within the original plurality of image lines, wherein the one or more isolated point targets are focused at one or more depths in an image, and wherein the phase sensitive summation comprises a phase sensitive vector summation of the at least two of the original plurality of image lines.

96. The method of claim 92, further comprising performing edge-based machine learning computations associated with an image or image analysis using an artificial intelligence algorithm to identify one or more of a tissue border, plaque, calcium, thrombus, dissection, and/or stent apposition.

97. A system for capturing and processing imaging data, the system comprising:
a rotating single element ultrasound transducer; and
a processor configured to be placed in communication with the rotating single element ultrasound transducer, the processor configured to:
acquire an original plurality of image lines from the rotating single element ultrasound transducer,
wherein each of the original plurality of image lines is acquired by a pulse-echo emission and a pulse-echo reception while the rotating single element ultrasound transducer is spinning, and
wherein each of the original plurality of image lines corresponds to an angular directivity; and
process the original plurality of image lines to produce a modified plurality of image lines with modified angular directivity characteristics via:
a modification by one or more of: scaling of an amplitude, a phase rotation, and a time shift, and
a phase sensitive summation of at least two of the original plurality of image lines to produce each of the modified plurality of image lines,
wherein the modification by one or more of: the scaling of the amplitude, the phase rotation, and the time shift is configured to produce the modified plurality of image lines.
